(19) United States
(12) Patent Application Publication
(10) Pub. No.: US 2002/0104105 A1
(43) Pub. Date: Aug. 1, 2002

(54) Protein-protein interactions

(75) Inventors: Karen Heichman, Salt Lake City, UT (US); Paul L. Bartel, Salt Lake City, UT (US)

Correspondence Address: ROTHWELL, FIGG, ERNST & MANBECK, P.C., 555 13TH STREET, N.W., SUITE 701, EAST TOWER, WASHINGTON, DC, 20004, US

(73) Assignee: MYRIAD GENETICS, INC., Salt Lake City, UT

(21) Appl. No.: 09/885,535

(22) Filed: Jun. 21, 2001

Related U.S. Application Data
(60) Provisional application No. 60/213,245, filed on Jun. 22, 2000.

(51) Int. Cl. A01K 67/00; G01N 33/53; G01N 33/537; G01N 33/543; C07K 14/435

(52) U.S. Cl. 800/8; 530/350; 530/388.1; 435/7.92

(57) ABSTRACT

The present invention relates to the discovery of novel protein-protein interactions that are involved in mammalian physiological pathways, including physiological disorders or diseases. Examples of physiological disorders and diseases include non-insulin dependent diabetes mellitus (NIDDM), neurodegenerative disorders, such as Alzheimer's Disease (AD), and the like. Thus, the present invention is directed to complexes of these proteins and/or their fragments, antibodies to the complexes, diagnosis of physiological generative disorders (including diagnosis of a predisposition to and diagnosis of the existence of the disorder), drug screening for agents which modulate the interaction of proteins described herein, and identification of additional proteins in the pathway common to the proteins described herein.

### CROSS-REFERENCE TO RELATED APPLICATION

The present application is related to U.S. provisional patent application Ser. No. 60/213,245 filed on Jun. 22, 2000, incorporated herein by reference.

### BACKGROUND OF THE INVENTION

The present invention relates to the discovery of novel protein-protein interactions that are involved in mammalian physiological pathways, including physiological disorders or diseases. Examples of physiological disorders and diseases include non-insulin dependent diabetes mellitus (NIDDM), neurodegenerative disorders, such as Alzheimer's Disease (AD), and the like. Thus, the present invention is directed to complexes of these proteins and/or their fragments, antibodies to the complexes, diagnosis of physiological generative disorders (including diagnosis of a predisposition to and diagnosis of the existence of the disorder), drug screening for agents which modulate the interaction of proteins described herein, and identification of additional proteins in the pathway common to the proteins described herein.

The publications and other materials used herein to illuminate the background of the invention, and in particular, cases to provide additional details respecting the practice, are incorporated herein by reference, and for convenience, are referenced by author and date in the following text and respectively grouped in the appended Bibliography.

Many processes in biology, including transcription, translation and metabolic or signal transduction pathways, are mediated by non-covalently associated protein complexes. The formation of protein-protein complexes or protein-DNA complexes produce the most efficient chemical machinery. Much of modem biological research is concerned with identifying proteins involved in cellular processes, determining their functions, and how, when and where they interact with other proteins involved in specific pathways. Further, with rapid advances in genome sequencing, there is a need to define protein linkage maps, i.e., detailed inventories of protein interactions that make up functional assemblies of proteins or protein complexes or that make up physiological pathways.

Recent advances in human genomics research has led to rapid progress in the identification of novel genes. In applications to biological and pharmaceutical research, there is a need to determine functions of gene products. A first step in defining the function of a novel gene is to determine its interactions with other gene products in appropriate context. That is, since proteins make specific interactions with other proteins or other biopolymers as part of functional assemblies or physiological pathways, an appropriate way to examine function of a gene is to determine its physical relationship with other genes. Several systems exist for identifying protein interactions and hence relationships between genes.

There continues to be a need in the art for the discovery of additional protein-protein interactions involved in mammalian physiological pathways. There continues to be a need in the art also to identify the protein-protein interactions that are involved in mammalian physiological disorders and diseases, and to thus identify drug targets.

### SUMMARY OF THE INVENTION

The present invention relates to the discovery of protein-protein interactions that are involved in mammalian physiological pathways, including physiological disorders or diseases, and to the use of this discovery. The identification of the interacting proteins described herein provide new targets for the identification of useful pharmaceuticals, new targets for diagnostic tools in the identification of individuals at risk, sequences for production of transformed cell lines, cellular models and animal models, and new bases for therapeutic intervention in such physiological pathways

Thus, one aspect of the present invention is protein complexes. The protein complexes are a complex of (a) two interacting proteins, (b) a first interacting protein and a fragment of a second interacting protein, (c) a fragment of a first interacting protein and a second interacting protein, or (d) a fragment of a first interacting protein and a fragment of a second interacting protein. The fragments of the interacting proteins include those parts of the proteins, which interact to form a complex. This aspect of the invention includes the detection of protein interactions and the production of proteins by recombinant techniques. The latter embodiment also includes cloned sequences, vectors, transfected or transformed host cells and transgenic animals.

A second aspect of the present invention is an antibody that is immunoreactive with the above complex. The antibody may be a polyclonal antibody or a monoclonal antibody. While the antibody is immunoreactive with the complex, it is not immunoreactive with the component parts of the complex. That is, the antibody is not immunoreactive with a first interactive protein, a fragment of a first interacting protein, a second interacting protein or a fragment of a second interacting protein. Such antibodies can be used to detect the presence or absence of the protein complexes.

A third aspect of the present invention is a method for diagnosing a predisposition for physiological disorders or diseases in a human or other animal. The diagnosis of such disorders includes a diagnosis of a predisposition to the disorders and a diagnosis for the existence of the disorders. In accordance with this method, the ability of a first interacting protein or fragment thereof to form a complex with a second interacting protein or a fragment thereof is assayed, or the genes encoding interacting proteins are screened for mutations in interacting portions of the protein molecules. The inability of a first interacting protein or fragment thereof to form a complex, or the presence of mutations in a gene within the interacting domain, is indicative of a predisposition to, or existence of a disorder. In accordance with one embodiment of the invention, the ability to form a complex is assayed in a two-hybrid assay. In a first aspect of this embodiment, the ability to form a complex is assayed by a yeast two-hybrid assay. In a second aspect, the ability to form a complex is assayed by a mammalian two-hybrid assay. In a second embodiment, the ability to form a complex is assayed by measuring in vitro a complex formed by combining said first protein and said second protein. In one aspect the proteins are isolated from a human or other animal. In a third embodiment, the ability to form a complex is assayed by measuring the binding of an antibody, which is specific for the complex. In a fourth embodiment, the ability to form a complex is assayed by measuring the binding of an antibody that is specific for the complex with a tissue extract from a human or other animal. In a fifth embodiment, coding sequences of the interacting proteins described herein are screened for mutations.

A fourth aspect of the present invention is a method for screening for drug candidates which are capable of modulating the interaction of a first interacting protein and a second interacting protein. In this method, the amount of the complex formed in the presence of a drug is compared with the amount of the complex formed in the absence of the drug. If the amount of complex formed in the presence of the drug is greater than or less than the amount of complex formed in the absence of the drug, the drug is a candidate for modulating the interaction of the first and second interacting proteins. The drug promotes the interaction if the complex formed in the presence of the drug is greater and inhibits (or disrupts) the interaction if the complex formed in the presence of the drug is less. The drug may affect the interaction directly, i.e., by modulating the binding of the two proteins, or indirectly, e.g., by modulating the expression of one or both of the proteins.

A fifth aspect of the present invention is a model for such physiological pathways, disorders or diseases. The model may be a cellular model or an animal model, as further described herein. In accordance with one embodiment of the invention, an animal model is prepared by creating transgenic or “knock-out” animals. The knock-out may be a total knock-out, i.e., the desired gene is deleted, or a conditional knock-out, i.e., the gene is active until it is knocked out at a determined time. In a second embodiment, a cell line is derived from such animals for use as a model. In a third embodiment, an animal model is prepared in which the biological activity of a protein complex of the present invention has been altered. In one aspect, the biological activity is altered by disrupting the formation of the protein complex, such as by the binding of an antibody or small molecule to one of the proteins which prevents the formation of the protein complex. In a second aspect, the biological activity of a protein complex is altered by disrupting the action of the complex, such as by the binding of an antibody or small molecule to the protein complex which interferes with the action of the protein complex as described herein. In a fourth embodiment, a cell model is prepared by altering the genome of the cells in a cell line. In one aspect, the genome of the cells is modified to produce at least one protein complex described herein. In a second aspect, the genome of the cells is modified to eliminate at least one protein of the protein complexes described herein.

A sixth aspect of the present invention are nucleic acids coding for novel proteins discovered in accordance with the present invention and the corresponding proteins and antibodies.

A seventh aspect of the present invention is a method of screening for drug candidates useful for treating a physiological disorder. In this embodiment, drugs are screened on the basis of the association of a protein with a particular physiological disorder. This association is established in accordance with the present invention by identifying a relationship of the protein with a particular physiological disorder. The drugs are screened by comparing the activity of the protein in the presence and absence of the drug. If a difference in activity is found, then the drug is a drug candidate for the physiological disorder. The activity of the protein can be assayed in vitro or in vivo using conventional techniques, including transgenic animals and cell lines of the present invention.

### DETAILED DESCRIPTION OF THE INVENTION

The present invention is the discovery of novel interactions between proteins described herein. The genes coding for some of these proteins may have been cloned previously, but their potential interaction in a physiological pathway or with a particular protein was unknown. Alternatively, the genes coding for some of these proteins have not been cloned previously and represent novel genes. These proteins are identified using the yeast two-hybrid method and searching a human total brain library, as more fully described below.

According to the present invention, new protein-protein interactions have been discovered. The discovery of these interactions has identified several protein complexes for each protein-protein interaction. The protein complexes for these interactions are set forth below in Tables 1-12, which also identify the new protein-protein interactions of the present invention.

TABLE 1
Protein Complexes of MAPKAP-K3/AP-3 Delta Interaction

MAP Kinase MAPKAP-K3 (MAPKAP-K3) and AP-3 Delta
A fragment of MAPKAP-K3 and AP-3 Delta
MAPKAP-K3 and a fragment of AP-3 Delta
A fragment of MAPKAP-K3 and a fragment of AP-3 Delta

TABLE 2
Protein Complexes of MAPKAP-K3/APP-695 Interaction

MAP Kinase MAPKAP-K3 (MAPKAP-K3) and Amyloid Aβ Precursor
Protein (APP-695)
A fragment of MAPKAP-K3 and APP-695
MAPKAP-K3 and a fragment of APP-695
A fragment of MAPKAP-K3 and a fragment of APP-695

TABLE 3
Protein Complexes of MAPKAP-K3/Hsp8 Interaction

MAP Kinase MAPKAP-K3 (MAPKAP-K3) and Heat Shock Protein 8
(Hsp8)
A fragment of MAPKAP-K3 and Hsp8
MAPKAP-K3 and a fragment of Hsp8
A fragment of MAPKAP-K3 and a fragment of Hsp8

TABLE 4
Protein Complexes of L130/NY-REN-58 Interaction

Leucine Rich Protein L130 (L130) and NY-REN-58
A fragment of L130 and NY-REN-58
L130 and a fragment of NY-REN-58
A fragment of L130 and a fragment of NY-REN-58

TABLE 5
Protein Complexes of P38 Alpha/P38 Beta Interaction

Protein Kinase p38 alpha (p38 alpha) and Protein Kinase p38 beta
(p38 beta)
A fragment of p38 alpha and p38 beta
p38 alpha and a fragment of p38 beta
A fragment of p38 alpha and a fragment of p38 beta

TABLE 6
Protein Complexes of ERK3/KIAA0934 Interaction

ERK3 and KIAA0934
A fragment of ERK3 and KIAA0934
ERK3 and a fragment of KIAA0934
A fragment of ERK3 and a fragment of KIAA0934

TABLE 7
Protein Complexes of ERK3/CDK9 Interaction

ERK3 and CDK9
A fragment of ERK3 and CDK9
ERK3 and a fragment of CDK9
A fragment of ERK3 and a fragment of CDK9

TABLE 8
Protein Complexes of ERK3/CLK Interaction

ERK3 and Clk Protein Kinase (CLK)
A fragment of ERK3 and CLK
ERK3 and a fragment of CLK
A fragment of ERK3 and a fragment of CLK

TABLE 9
Protein Complexes of C-NAP-1/Clathrin HC Interaction

C-NAP-1 and Clathrin Heavy Chain (Clathrin HC)
A fragment of C-NAP-1 and Clathrin HC
C-NAP-1 and a fragment of Clathrin HC
A fragment of C-NAP-1 and a fragment of Clathrin HC

TABLE 10
Protein Complexes of C-NAP-1/Amphiphysin Interaction

C-NAP-1 and Amphiphysin
A fragment of C-NAP-1 and Amphiphysin
C-NAP-1 and a fragment of Amphiphysin
A fragment of C-NAP-1 and a fragment of Amphiphysin

TABLE 11
Protein Complexes of C-NAP-1/PN9109 Interaction

C-NAP-1 and Novel Protein 9109 (PN9109)
A fragment of C-NAP-1 and PN9109
C-NAP-1 and a fragment of PN9109
A fragment of C-NAP-1 and a fragment of PN9109

TABLE 12
Protein Complexes of C-NAP-1/KIAA1106 Interaction

C-NAP-1 and KIAA1106
A fragment of C-NAP-1 and KIAA1106
C-NAP-1 and a fragment of KIAA1106
A fragment of C-NAP-1 and a fragment of KIAA1106

The involvement of above interactions in particular pathways is as follows.

Many cellular proteins exert their function by interacting with other proteins in the cell. Examples of this are found in the formation of multiprotein complexes and the association of enzymes with their substrates. It is widely believed that a great deal of information can be gained by understanding individual protein-protein interactions, and that this is useful in identifying complex networks of interacting proteins that participate in the workings of normal cellular functions. Ultimately, the knowledge gained by characterizing these networks can lead to valuable insight into the causes of human diseases and can eventually lead to the development of therapeutic strategies. The yeast two-hybrid assay is a powerful tool for determining protein-protein interactions and it has been successfully used for studying human disease pathways. In one variation of this technique, a protein of interest (or a portion of that protein) is expressed in a population of yeast cells that collectively contain all protein sequences. Yeast cells that possess protein sequences that interact with the protein of interest are then genetically selected, and the identity of those interacting proteins are determined by DNA sequencing. Thus, proteins that can be demonstrated to interact with a protein known to be involved in a human disease are therefore also implicated in that disease. To create a more complex network of interactions in a disease pathway, proteins which were identified in the first round of two-hybrid screening are subsequently used in two-hybrid assays as the protein of interest.

Cellular events that are initiated by exposure to growth factors, cytokines and stress are propagated from the outside of the cell to the nucleus by means of several protein kinase signal transduction cascades. p38 kinase is a member of the MAP kinase family of protein kinases. It is a key player in signal transduction pathways induced by the proinflammatory cytokines such as tumor necrosis factor (TNF), interleukin-1 (IL-1) and interleukin-6 (IL-6) and it also plays a critical role in the synthesis and release of the proinflammatory cytokines (Raingeaud et al., 1995; Lee et al., 1996; Miyazawa et al., 1998; Lee et al., 1994). Studies of inhibitors of p38 kinase have shown that blocking p38 kinase activity can cause anti-inflammatory effects, thus suggesting that this may be a mechanism of treating certain inflammatory diseases such as rheumatoid arthritis and inflammatory bowel disease. Further, p38 kinase activity has been implicated in other human diseases such as atherosclerosis, cardiac hypertrophy and hypoxic brain injury (Grammer et al., 1998; Mach et al., 1998; Wang et al., 1998; Nemoto et al., 1998; Kawasaki et al., 1997). Thus, by understanding p38 function, one may gain novel insight into a cellular response mechanism that affects a number of tissues and can potentially lead to harmful affects when disrupted.

The search for the physiological substrates of p38 kinase has taken a number of approaches including a variety of biochemical and cell biological methods. There are four known human isoforms of p38 kinase termed alpha, beta, gamma and delta, and these are thought to possess different physiological functions, likely because they have distinct substrate and tissue specificities. Some of the p38 kinase substrates are known, and the list includes transcription factors and additional protein kinases that act downstream of p38 kinase. Four of the kinases that act downstream of p38 kinase, MAPKAP-K2, MAPKAP-K3, PRAK and MSK1, are currently being analyzed themselves and some of their substrates have been identified.

The yeast two-hybrid system has been used to detect potential substrates and upstream regulators of the p38 kinases and their downstream kinases. In a two-hybrid search using p38 alpha kinase as the protein of interest, the highly related p38 beta kinase was shown to bind to p38 alpha. p38 beta kinase is 74% identical to p38 alpha, however it responds differently to upstream kinases and some extracellular stimuli (Jiang et al., 1996). The finding that p38 alpha and p38 beta interact could be interpreted in a number or ways. For one, it is possible that p38 alpha or beta can utilize the other as a substrate for its kinase activity. Alternatively, it is possible that the regions of p38 alpha and beta, the N-terminal and C-terminal portions, respectively, interact with one another to mimic the normal intracellular contacts that occur in protein folding. Nonetheless, this result is interesting since it suggests that the activity of each of these kinases may be mediated by introducing fragments of the other.

MAPKAP-K3, a protein kinase that acts downstream of p38 kinase in the same signal transduction pathway, was used in a two-hybrid search to identify potential substrates or regulators. MAPKAP-K3 was demonstrated to interact with three proteins in the yeast two-hybrid assay. The first protein is the AP-3 delta protein trafficking factor. AP-3 delta is a subunit of the AP-3 adaptor-like complex that is involved in the transport of transmembrane proteins (Simpson et al., 1997). AP-3 delta itself contains a single putative transmembrane domain towards the middle of the protein and 3 predicted MAPKAP phosphorylation sites in the C-terminal half. Since the MAPKAP phosphorylation sites of AP-3 delta all reside within the C-terminal side, one is tempted to speculate that the N-terminus of AP-3 delta is oriented toward the inside of a transport vesicle while the C-terminus is exposed to the cytoplasm where it could contact MAPKAP-K3 and be utilized as one of its substrates.

The second protein shown to interact with MAPKAP-K3 is the amyloid A-beta precursor protein (APP-695). APP-695 is a type I membrane protein that is proteolytically processed to yield a secreted form of the protein. The region of APP-695 that interacts with MAPKAP-K3 in the two-hybrid assay (amino acids 409 to 550) lies in the extracellular portion of the protein, therefore it is a bit difficult to ascertain the biological significance of this association.

The third protein demonstrated to interact with MAPKAP-K3 is the Hsp8 70 kD protein (Hsc70). MAPKAP-K3 has been previously shown to bind to another heat shock protein Hsp27, and it has been demonstrated that Hsp27 is a phosphorylation substrate of MAPKAP-K3 (Clifton et al., 1996). Hsp8 may also be capable of being phosphorylated by the MAPKAPs since it contains a putative MAPKAP consensus phosphorylation site. Interestingly, Hsp8 has been implicated in the regulation of AP-1 responsive genes by virtue of its ability to affect the DNA-binding activity of AP-1 in in vitro studies (Carter, 1997). Thus, the finding that MAPKAP-K3 associates with Hsp8 may provide yet another link between the MAPKAPs and the transcriptional induction in response to cellular and physiological stress.

Yeast two-hybrid searches have been performed using a leucine-rich protein of unknown function called L130 that was previously identified by us to be a common interactor of both MAPKAP-K2 and PRAK. L130 was originally identified by virtue of its high level of expression in hepatoblastoma cells (Hou et al., 1994), however there is currently no information about its function. Its expression in hepatoblastoma cells suggests a role in liver function or in the transformation of normal cells to malignant ones. L130 has been shown to interact with a protein called NY-REN-58. NY-REN-58 was isolated as an antigen that was recognized by an antibody found in renal-cell carcinoma patients (Scanlan et al., 1999). There do not appear to be any obvious structural domains present in NY-REN-58, however it does possess some sequence similarity to the coiled-coil containing centromere protein F.

In our previous findings, the ERK3 protein kinase was shown to interact with PRAK. ERK3 is a serine/threonine protein kinase of relatively unknown function (Cheng et al., 1996). It is a nuclear protein present in several tissues and is expressed in response to a number of extracellular stimuli. In two-hybrid searches using ERK3 as a protein of interest, three proteins were shown to be interactors. The first protein, the cell cycle-dependent kinase, CDK9, also known as PITARLE, is a CDC2-related serine/threonine protein kinase that is ubiquitously expressed and localized to the nucleus (Grana et al., 1994; Best et al., 1995). CDK9 complexes with at least three different cyclins (Fu et al., 1999; Bieniasz et al., 1998) and appears to have a number of in vitro substrates which include the retinoblastoma and myelin basic proteins. It has been shown that CDK9 is the catalytic subunit of a multi-protein complex called the P-TEFb (positive transcription elongation factor b) that phosphorylates and activates the C-terminal domain of the large subunit of RNA polymerase II (Zhu et al., 1997). Interestingly, P-TEFb has been shown to be the HIV Tat-associated kinase (TAK) that is induced by the activation of peripheral blood lymphocytes and differentiation of promonocytic cell lines (Yang et al., 1997). The finding that ERK3 interacts with CDK9 suggests that ERK3 may be capable of phosphorylating CDK9, or vice versa. In support of this notion, CDK9 appears to contain 3 consensus MAP kinase phsophorylation sites. Interestingly, CDK9 has also been shown to interact with TRAF2 (tumor necrosis factor signal transducer) that is thought to act as a cytoplasmic linker protein (MacLachlan et al., 1998). This is yet another tie between CDK9 and the inflammation response.

The second protein found to interact with ERK3 is the Clk protein kinase. Clk (also known as Sty) was originally cloned by virtue of its similarity to the yeast cdc2/CDC28 protein kinase (Johnson and Smith, 1991). Unlike the cyclin-dependent kinases which are specific for serine and threonine residues, CLK is a dual specificity protein kinase that phosphorylates serines, threonines and tyrosines. CLk localizes to the nucleus and has been shown to phosphorylate the SR serine/arginine-rich splicing factors (Colwill et al., 1996). In fact, Clk has also been demonstrated to modulate SR protein splicing activity in both in vivo and in vitro assays (Prasad et al., 1999). The finding that ERK3 and Clk associate with one another suggests that either ERK3 is a substrate of Clk, or that Clk is a substrate of ERK3. If ERK3 is capable of phosphorylating Clk, then ERK3 may linked to the regulation of splicing via its modulation of Clk activity.

The third protein shown to interact with ERK3 is a portion of a protein fragment of unknown function was shown to be an interactor. This sequence is called KIAA0934 and has no incriminating features other than a single predicted transmembrane domain, a beta/gamma crystallin motif and a MAP kinase consensus phosphorylation site. A brief survey of ESTs indicates that KIAA0934 is expressed in a wide variety of tissues. KIAA0934 is similar to KIAA0184 (GenBank entry D80006) that also has no known function. Since KIAA0934 was isolated as an interactor of ERK3 and because its protein sequence appears to have a MAP kinase phosphorylation site, it is possible that KIAA0934 can act as a substrate for ERK3.

Yeast two-hybrid assays have been performed using the C-NAP1 protein that was previously identified by us as an interactor of the p38 alpha kinase and was also shown to interact with the Nek2 cell cycle-regulated protein kinase in studies performed by others (Fry et al., 1998). In this study, we have shown that C-NAP1 interacts with four proteins. Two proteins involved in vesicular transport were shown to be interactors of C-NAP 1. The first protein is the clathrin heavy chain, the major protein of the clathrin coated pit involved in endocytosis (Ybe et al., 1999). The region of the clathrin heavy chain that binds to C-NAP1 corresponds to the so-called proximal segment and is directly adjacent to the portion of clathrin heavy chain that interacts with the clathrin light chain. In two-hybrid studies reported in the literature, clathrin heavy chain has been shown to bind to the guanine nucleotide exchange factor p532 (Rosa et al., 1997). The second protein involved in vesicular transport shown to be an interactor of amino acids 25 to 93 of C-NAP1 is called amphiphysin. Amphiphysin is an SH3 domain-containing protein that associates with the cytoplasmic surface of synaptic vesicles and has been implicated in clathrin-mediated endocytosis (Takei et la. 1999). Taken together, these results strongly suggest that C-NAP1 itself plays a role in vesicular transport. In other studies performed by Myriad Genetics, Inc., amphiphysin has been demonstrated to interact with the APC (adenomatous polyposis coli) tumor suppressor, the BAI3 angiogenesis inhibitor as well as the PI3 kinase p110 gamma subunit. Thus, amphiphysin, and C-NAP1 by inference, may play a role in cancer or angiogenesis. Since C-NAP1 has been previously shown to interact with two protein kinases, NEK2 and p38 alpha, it seems possible that C-NAP1 function may be regulated by protein phosphorylation.

Two proteins of unknown function have also been shown to associate with C-NAP1 in the yeast two-hybrid assay. The first interactor is a novel sequence called PN9109 (sequence disclosed herein). There may be some clues to be had with regard to its cellular role. First, although the known protein sequence is still incomplete, PN9109 is 2835 amino acids in length so far and contains two EF hand calcium-binding motifs; additionally, PN9109 also appears to be an alternative splice of the KIAA0728 gene (GenBank entry AB018271). Second, PN9109 is very similar to the ABP620 actin-binding protein that was shown in previous studies to interact with PRAK. PRAK and C-NAP1 share the p38 alpha kinase as a two-hybrid interactor, suggesting that there may be some important multiprotein complex that includes PN9109, C-NAP 1, PRAK and p38 alpha kinase. Perhaps PN9109 and C-NAP1 serve to provide a link between transport vesicles and actin filaments.

The second protein of no known function shown to interact with C-NAP1 is called KIAA1106. KIAA1106 does not appear to have any distinguishing domains that lend insight into this area. The one clue to its cellular role lies in the fact that it bears sequence similarity to MTF 1 (myelin transcription factor), another protein that was identified also as an interactor of C-NAP1. Interestingly, KIAA1106 and MTF 1 interact with the same region of C-NAP 1.

The proteins disclosed in the present invention were found to interact with their corresponding proteins in the yeast two-hybrid system. Because of the involvement of the corresponding proteins in the physiological pathways disclosed herein, the proteins disclosed herein also participate in the same physiological pathways. Therefore, the present invention provides a list of uses of these proteins and DNA encoding these proteins for the development of diagnostic and therapeutic tools useful in the physiological pathways. This list includes, but is not limited to, the following examples.

Two-hybrid System

The principles and methods of the yeast two-hybrid system have been described in detail elsewhere (e.g., Bartel and Fields, 1997; Bartel et al., 1993; Fields and Song, 1989; Chevray and Nathans, 1992). The following is a description of the use of this system to identify proteins that interact with a protein of interest.

The target protein is expressed in yeast as a fusion to the DNA-binding domain of the yeast Gal4p. DNA encoding the target protein or a fragment of this protein is amplified from cDNA by PCR or prepared from an available clone. The resulting DNA fragment is cloned by ligation or recombination into a DNA-binding domain vector (e.g., pGBT9, pGBT.C, pAS2-1) such that an in-frame fusion between the Gal4p and target protein sequences is created.

The target gene construct is introduced, by transformation, into a haploid yeast strain. A library of activation domain fusions (i.e., adult brain cDNA cloned into an activation domain vector) is introduced by transformation into a haploid yeast strain of the opposite mating type. The yeast strain that carries the activation domain constructs contains one or more Gal4p-responsive reporter gene(s), whose expression can be monitored. Examples of some yeast reporter strains include Y190, PJ69, and CBY14a. An aliquot of yeast carrying the target gene construct is combined with an aliquot of yeast carrying the activation domain library. The two yeast strains mate to form diploid yeast and are plated on media that selects for expression of one or more Gal4p-responsive reporter genes. Colonies that arise after incubation are selected for further characterization.

The activation domain plasmid is isolated from each colony obtained in the two-hybrid search. The sequence of the insert in this construct is obtained by the dideoxy nucleotide chain termination method. Sequence information is used to identify the gene/protein encoded by the activation domain insert via analysis of the public nucleotide and protein databases. Interaction of the activation domain fusion with the target protein is confirmed by testing for the specificity of the interaction. The activation domain construct is co-transformed into a yeast reporter strain with either the original target protein construct or a variety of other DNA-binding domain constructs. Expression of the reporter genes in the presence of the target protein but not with other test proteins indicates that the interaction is genuine.

In addition to the yeast two-hybrid system, other genetic methodologies are available for the discovery or detection of protein-protein interactions. For example, a mammalian two-hybrid system is available commercially (Clontech, Inc.) that operates on the same principle as the yeast two-hybrid system. Instead of transforming a yeast reporter strain, plasmids encoding DNA-binding and activation domain fusions are transfected along with an appropriate reporter gene (e.g., lacZ) into a mammalian tissue culture cell line. Because transcription factors such as the Saccharomyces cerevisiae Gal4p are functional in a variety of different eukaryotic cell types, it would be expected that a two-hybrid assay could be performed in virtually any cell line of eukaryotic origin (e.g., insect cells (SF9), fungal cells, worm cells, etc.). Other genetic systems for the detection of protein-protein interactions include the so-called SOS recruitment system (Aronheim et al., 1997).

Protein-protein Interactions

Protein interactions are detected in various systems including the yeast two-hybrid system, affinity chromatography, co-immunoprecipitation, subcellular fractionation and isolation of large molecular complexes. Each of these methods is well characterized and can be readily performed by one skilled in the art. See, e.g., U.S. Pat. Nos. 5,622,852 and 5,773,218, and PCT published application No. WO 97/27296 and PCT published application No. WO 99/65939, each of which are incorporated herein by reference.

The protein of interest can be produced in eukaryotic or prokaryotic systems. A cDNA encoding the desired protein is introduced in an appropriate expression vector and transfected in a host cell (which could be bacteria, yeast cells, insect cells, or mammalian cells). Purification of the expressed protein is achieved by conventional biochemical and immunochemical methods well known to those skilled in the art. The purified protein is then used for affinity chromatography studies: it is immobilized on a matrix and loaded on a column. Extracts from cultured cells or homogenized tissue samples are then loaded on the column in appropriate buffer, and non-binding proteins are eluted. After extensive washing, binding proteins or protein complexes are eluted using various methods such as a gradient of pH or a gradient of salt concentration. Eluted proteins can then be separated by two-dimensional gel electrophoresis, eluted from the gel, and identified by micro-sequencing. The purified proteins can also be used for affinity chromatography to purify interacting proteins disclosed herein. All of these methods are well known to those skilled in the art.

Similarly, both proteins of the complex of interest (or interacting domains thereof) can be produced in eukaryotic or prokaryotic systems. The proteins (or interacting domains) can be under control of separate promoters or can be produced as a fusion protein. The fusion protein may include a peptide linker between the proteins (or interacting domains) which, in one embodiment, serves to promote the interaction of the proteins (or interacting domains). All of these methods are also well known to those skilled in the art.

Purified proteins of interest, individually or a complex, can also be used to generate antibodies in rabbit, mouse, rat, chicken, goat, sheep, pig, guinea pig, bovine, and horse. The methods used for antibody generation and characterization are well known to those skilled in the art. Monoclonal antibodies are also generated by conventional techniques. Single chain antibodies are further produced by conventional techniques.

DNA molecules encoding proteins of interest can be inserted in the appropriate expression vector and used for transfection of eukaryotic cells such as bacteria, yeast, insect cells, or mammalian cells, following methods well known to those skilled in the art. Transfected cells expressing both proteins of interest are then lysed in appropriate conditions, one of the two proteins is immunoprecipitated using a specific antibody, and analyzed by polyacrylamide gel electrophoresis. The presence of the binding protein (co-immunoprecipitated) is detected by immunoblotting using an antibody directed against the other protein. Co-immunoprecipitation is a method well known to those skilled in the art.

Transfected eukaryotic cells or biological tissue samples can be homogenized and fractionated in appropriate conditions that will separate the different cellular components. Typically, cell lysates are run on sucrose gradients, or other materials that will separate cellular components based on size and density. Subcellular fractions are analyzed for the presence of proteins of interest with appropriate antibodies, using immunoblotting or immunoprecipitation methods. These methods are all well known to those skilled in the art.

Disruption of Protein-protein Interactions

It is conceivable that agents that disrupt protein-protein interactions can be beneficial in many physiological disorders, including, but not-limited to NIDDM, AD and others disclosed herein. Each of the methods described above for the detection of a positive protein-protein interaction can also be used to identify drugs that will disrupt said interaction. As an example, cells transfected with DNAs coding for proteins of interest can be treated with various drugs, and co-immunoprecipitations can be performed. Alternatively, a derivative of the yeast two-hybrid system, called the reverse yeast two-hybrid system (Leanna and Hannink, 1996), can be used, provided that the two proteins interact in the straight yeast two-hybrid system.

Modulation of Protein-protein Interactions

Since the interaction described herein is involved in a physiological pathway, the identification of agents which are capable of modulating the interaction will provide agents which can be used to track the physiological disorder or to use as lead compounds for development of therapeutic agents. An agent may modulate expression of the genes of interacting proteins, thus affecting interaction of the proteins. Alternatively, the agent may modulate the interaction of the proteins. The agent may modulate the interaction of wild-type with wild-type proteins, wild-type with mutant proteins, or mutant with mutant proteins. Agents can be tested using transfected host cells, cell lines, cell models or animals, such as described herein, by techniques well known to those of ordinary skill in the art, such as disclosed in U.S. Pat. Nos. 5,622,852 and 5,773,218, and PCT published application No. WO 97/27296 and PCT published application No. WO 99/65939, each of which are incorporated herein by reference. The modulating effect of the agent can be screened in vivo or in vitro. Exemplary of a method to screen agents is to measure the effect that the agent has on the formation of the protein complex.

Mutation Screening

The proteins disclosed in the present invention interact with one or more proteins known to be involved in a physiological pathway, such as in NIDDM or AD. Mutations in interacting proteins could also be involved in the development of the physiological disorder, such as NIDDM or AD, for example, through a modification of protein-protein interaction, or a modification of enzymatic activity, modification of receptor activity, or through an unknown mechanism. Therefore, mutations can be found by sequencing the genes for the proteins of interest in patients having the physiological disorder, such as insulin, and non-affected controls. A mutation in these genes, especially in that portion of the gene involved in protein interactions in the physiological pathway, can be used as a diagnostic tool and the mechanistic understanding the mutation provides can help develop a therapeutic tool.

Screening for At-risk Individuals

Individuals can be screened to identify those at risk by screening for mutations in the protein disclosed herein and identified as described above. Alternatively, individuals can be screened by analyzing the ability of the proteins of said individual disclosed herein to form natural complexes. Further, individuals can be screened by analyzing the levels of the complexes or individual proteins of the complexes or the mRNA encoding the protein members of the complexes. Techniques to detect the formation of complexes, including those described above, are known to those skilled in the art. Techniques and methods to detect mutations are well known to those skilled in the art. Techniques to detect the level of the complexes, proteins or mRNA are well known to those skilled in the art.

Cellular Models of Physiological Disorders

A number of cellular models of many physiological disorders or diseases have been generated. The presence and the use of these models are familiar to those skilled in the art. As an example, primary cell cultures or established cell lines can be transfected with expression vectors encoding the proteins of interest, either wild-type proteins or mutant proteins. The effect of the proteins disclosed herein on parameters relevant to their particular physiological disorder or disease can be readily measured. Furthermore, these cellular systems can be used to screen drugs that will influence those parameters, and thus be potential therapeutic tools for the particular physiological disorder or disease. Alternatively, instead of transfecting the DNA encoding the protein of interest, the purified protein of interest can be added to the culture medium of the cells under examination, and the relevant parameters measured.

Animal Models

The DNA encoding the protein of interest can be used to create animals that overexpress said protein, with wild-type or mutant sequences (such animals are referred to as “transgenic”), or animals which do not express the native gene but express the gene of a second animal (referred to as “transplacement”), or animals that do not express said protein (referred to as “knock-out”). The knock-out animal may be an animal in which the gene is knocked out at a determined time. The generation of transgenic, transplacement and knock-out animals (normal and conditioned) uses methods well known to those skilled in the art.

In these animals, parameters relevant to the particular physiological disorder can be measured. These parametes may include receptor function, protein secretion in vivo or in vitro, survival rate of cultured cells, concentration of particular protein in tissue homogenates, signal transduction, behavioral analysis, protein synthesis, cell cycle regulation, transport of compounds across cell or nuclear membranes, enzyme activity, oxidative stress, production of pathological products, and the like. The measurements of biochemical and pathological parameters, and of behavioral parameters, where appropriate, are performed using methods well known to those skilled in the art. These transgenic, transplacement and knock-out animals can also be used to screen drugs that may influence the biochemical, pathological, and behavioral parameters relevant to the particular physiological disorder being studied. Cell lines can also be derived from these animals for use as cellular models of the physiological disorder, or in drug screening.

Rational Drug Design

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo.

Several approaches for use in rational drug design include analysis of three-dimensional structure, alanine scans, molecular modeling and use of anti-id antibodies. These techniques are well known to those skilled in the art.

Following identification of a substance which modulates or affects polypeptide activity, the substance may be further investigated. Furthermore, it may be manufactured and/or used in preparation, i.e., manufacture or formulation, or a composition such as a medicament, pharmaceutical composition or drug. These may be administered to individuals.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing are generally used to avoid randomly screening large numbers of molecules for a target property.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

Diagnostic Assays

The identification of the interactions disclosed herein enables the development of diagnostic assays and kits, which can be used to determine a predisposition to or the existence of a physiological disorder. In one aspect, one of the proteins of the interaction is used to detect the presence of a “normal” second protein (i.e., normal with respect to its ability to interact with the first protein) in a cell extract or a biological fluid, and further, if desired, to detect the quantitative level of the second protein in the extract or biological fluid. The absence of the “normal” second protein would be indicative of a predisposition or existence of the physiological disorder. In a second aspect, an antibody against the protein complex is used to detect the presence and/or quantitative level of the protein complex. The absence of the protein complex would be indicative of a predisposition or existence of the physiological disorder.

Nucleic Acids and Proteins

A nucleic acid or fragment thereof has substantial identity with another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. A protein or fragment thereof has substantial identity with another if, optimally aligned, there is an amino acid sequence identity of at least about 30% identity with an entire naturally-occurring protein or a portion thereof, usually at least about 70% identity, more ususally at least about 80% identity, preferably at least about 90% identity, and more preferably at least about 95% identity.

Identity means the degree of sequence relatedness between two polypeptide or two polynucleotides sequences as determined by the identity of the match between two strings of such sequences, such as the full and complete sequence. Identity can be readily calculated. While there exist a number of methods to measure identity between two polynucleotide or polypeptide sequences, the term “identity” is well known to skilled artisans (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Methods commonly employed to determine identity between two sequences include, but are not limited to those disclosed in Guide to Huge Computers, Martin J. Bishop, ed., Academic Press, San Diego, 1994, and Carillo, H., and Lipman, D., SIAM J Applied Math. 48:1073 (1988). Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (Devereux, J., et al., Nucleic Acids Research 12(1). 387 (1984)), BLASTP, BLASTN, FASTA (Altschul et al. (1990); Altschul et al. (1997)). The well-known Smith Waterman algorithm may also be used to determine identity.

As an illustration, by a polynucleotide having a nucleotide sequence having at least, for example, 95% “identity” to a reference nucleotide sequence of is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Alternatively, substantial homology or (similarity) exists when a nucleic acid or fragment thereof will hybridize to another nucleic acid (or a complementary strand thereof) under selective hybridization conditions, to a strand, or to its complement. Selectivity of hybridization exists when hybridization which is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known in the art. See, e.g., Asubel, 1992; Wetmur and Davidson, 1968.

Thus, as herein used, the term “stringent conditions” means hybridization will occur only if there is at least 95% and preferably at least 97% identity between the sequences. Such hybridization techniques are well known to those of skill in the art. Stringent hybridization conditions are as defined above or. alternatively, conditions under overnight incubation at 42° C. in a solution comprising: 50% formamide, 5× SSC (150 mM NaCl, 15 mM trisodium citrate), 50 nM sodium phosphate (pH7.6), 5× Denhardt's solution, 10% dextran sulfate, and 20 μg/ml denatured, sheared salmon sperm DNA, followed by washing the filters in 0.1× SSC at about 65° C.

The terms “isolated”, “substantially pure”, and “substantially homogeneous” are used interchangeably to describe a protein or polypeptide which has been separated from components which accompany it in its natural state. A monomeric protein is substantially pure when at least about 60 to 75% of a sample exhibits a single polypeptide sequence. A substantially pure protein will typically comprise about 60 to 90% W/W of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band upon staining the gel. For certain purposes, higher resolution may be provided by using HPLC or other means well known in the art which are utilized for purification.

Large amounts of the nucleic acids of the present invention may be produced by (a) replication in a suitable host or transgenic animals or (b) chemical synthesis using techniques well known in the art. Constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate which allow the protein to cross and/or lodge in cell membranes, and thus attain its functional topology, or be secreted from the cell. Such vectors may be prepared by means of standard recombinant techniques well known in the art.

### EXAMPLES

The present invention is further detailed in the following Examples, which are offered by way of illustration and are not intended to limit the invention in any manner. Standard techniques well known in the art or the techniques specifically described below are utilized.

### Example 1

### Yeast Two-hybrid System

The principles and methods of the yeast two-hybrid systems have been described in detail (Bartel and Fields, 1997). The following is thus a description of the particular procedure that we used, which was applied to all proteins.

The cDNA encoding the bait protein was generated by PCR from brain cDNA. Gene-specific primers were synthesized with appropriate tails added at their 5′ ends to allow recombination into the vector pGBTQ. The tail for the forward primer was 5′-GCAGGAAACAGCTATGACCATACAGTCAGCGGCCGCCACC-3′ (SEQ ID NO:1) and the tail for the reverse primer was 5′-ACGGCCAGTCGCGTGGAGTGTTATGTCATGCGGCCGCTA-3′ (SEQ ID NO:2). The tailed PCR product was then introduced by recombination into the yeast expression vector pGBTQ, which is a close derivative of pGBTC (Bartel et al., 1996) in which the polylinker site has been modified to include M13 sequencing sites. The new construct was selected directly in the yeast J693 for its ability to drive tryptophane synthesis (genotype of this strain: Mat α, ade2, his3, leu2, trp1, URA3::GAL1-lacZ LYS2::GAL1-HIS3 gal4del gal80del cyhR2). In these yeast cells, the bait is produced as a C-terminal fusion protein with the DNA binding domain of the transcription factor Gal4 (amino acids 1 to 147). A total human brain (37 year-old male Caucasian) cDNA library cloned into the yeast expression vector pACT2 was purchased from Clontech (human brain MATCHMAKER cDNA, cat. # HL4004AH), transformed into the yeast strain J692 (genotype of this strain: Mat a, ade2, his3, leu2, trp1, URA3::GAL1-lacZ LYS2::GAL1-HIS3 gal4del ga80del cyhR2), and selected for the ability to drive leucine synthesis. In these yeast cells, each cDNA is expressed as a fusion protein with the transcription activation domain of the transcription factor Gal4 (amino acids 768 to 881) and a 9 amino acid hemagglutinin epitope tag. J693 cells (Mat α type) expressing the bait were then mated with J692 cells (Mat α type) expressing proteins from the brain library. The resulting diploid yeast cells expressing proteins interacting with the bait protein were selected for the ability to synthesize tryptophan, leucine, histidine, and β-galactosidase. DNA was prepared from each clone, transformed by electroporation into E. coli strain KC8 (Clontech KC8 electrocompetent cells, cat. #C2023-1), and the cells were selected on ampicillin-containing plates in the absence of either tryptophane (selection for the bait plasmid) or leucine (selection for the brain library plasmid). DNA for both plasmids was prepared and sequenced by di-deoxynucleotide chain termination method. The identity of the bait cDNA insert was confirmed and the cDNA insert from the brain library plasmid was identified using BLAST program against public nucleotides and protein databases. Plasmids from the brain library (preys) were then individually transformed into yeast cells together with a plasmid driving the synthesis of lamin fused to the Gal4 DNA binding domain. Clones that gave a positive signal after β-galactosidase assay were considered false-positives and discarded. Plasmids for the remaining clones were transformed into yeast cells together with plasmid for the original bait. Clones that gave a positive signal after galactosidase assay were considered true positives.

### Example 2

### Identification of MAPKAP-K3/AP-3 Delta Interaction

A yeast two-hybrid system as described in Example 1 using amino acids encoded by nucleotides 433-1003 of MAPKAP-K3 (GenBank (GB) accession no. U09578) as bait was performed. One clone that was identified by this procedure included amino acids encoded by nucleotides 2023-2821 of AP-3 Delta (GB accession no. AF002163).

### Example 3

### Identification of MAPKAP-K3/APP-695 Interaction

A yeast two-hybrid system as described in Example 1 using amino acids encoded by nucleotides 433-1003 of MAPKAP-K3 (GB accession no. U09578) as bait was performed. One clone that was identified by this procedure included amino acids encoded by nucleotides 1349-1774 of APP-695 (GB accession no. X06989).

### Example 4

### Identification of MAPKAP-K3/Hsp8 Interaction

A yeast two-hybrid system as described in Example 1 using amino acids encoded by nucleotides 433-1003 of MAPKAP-K3 (GB accession no. U09578) as bait was performed. One clone that was identified by this procedure included amino acids 260-533 of Hsp8 (Swiss Protein (SP) accession no. P11142).

### Example 5

### Identification of L130/NY-REN-58 Interaction

A yeast two-hybrid system as described in Example 1 using amino acids 800-1100 of L130 (SP accession no. P42704) as bait was performed. One clone that was identified by this procedure included amino acids encoded by nucleotides 1262-2105 of NY-REN-58 (GB accession no. AF155115).

### Example 6

### Identification of p38 alpha/p38 beta Interaction

A yeast two-hybrid system as described in Example 1 using amino acids 1-130 of p38 alpha (SP accession no. Q13083) as bait was performed. One clone that was identified by this procedure included amino acids encoded by nucleotides 890-1110 of p38 beta (GB accession no. AF031135).

### Example 7

Identification of ERK3/KIAA0934 Interaction

A yeast two-hybrid system as described in Example 1 using amino acids 1-316 of ERK3 (SP accession no. Q16659) as bait was performed. One clone that was identified by this procedure included amino acids 1194-1352 of KIAA0934 (SP accession no. Q9YE4).

### Example 8

### Identification of ERK3/CDK9 Interaction

A yeast two-hybrid system as described in Example 1 using amino acids 1-316 of ERK3 (SP accession no. Q16659) as bait was performed. One clone that was identified by this procedure included amino acids 160-372 of CDK9 (SP accession no. 950750).

### Example 9

### Identification of ERK3/CLK Interaction

A yeast two-hybrid system as described in Example 1 using amino acids 1-316 of ERK3 (SP accession no. Q16659) as bait was performed. One clone that was identified by this procedure included amino acids 1-364 of CLK (SP accession no. P49759).

### Example 10

### Identification of C-NAP1/Clathrin HC Interaction

A yeast two-hybrid system as described in Example 1 using amino acids encoded by nucleotides 744-950 of C-NAP1 (GB accession no. AF049105) as bait was performed. One clone that was identified by this procedure included amino acids 865-1170 of Clathrin HC (SP accession no. Q00610).

### Example 11

Identification of C-NAP1/Amphiphysin Interaction

A yeast two-hybrid system as described in Example 1 using amino acids encoded by nucleotides 744-950 of C-NAP1 (GB accession no. AF049105) as bait was performed. One clone that was identified by this procedure included amino acids 93-273 of amphiphysin (SP accession no. P49418).

### Example 12

### Identification of C-NAP1/PN9109 Interaction

A yeast two-hybrid system as described in Example 1 using amino acids encoded by nucleotides 4421-533628-193 of C-NAP1 (GB accession no. AF049105) as bait was performed. One clone that was identified by this procedure included amino acids encoded by nucleotides 16-547 of novel protein PN 9109. The DNA sequence (SEQ ID NO:3) and the predicted protein sequence (SEQ ID NO:4) for PN9109 are set forth in Tables 13 and 14, respectively.

TABLE 13
Nucleotide Sequence of PN9109

tggcttgtggaaaaagaacttatggtcagtgttcttgggcccttgtcaattgacccaaat
atgctaaacacacaaaggcagcaggtgcagattttgctgcaagaattcgccactcggaaa
cctcaatatgaacagctgacagcagctggtcagggcattctgagcaggcctggagaagac
ccttctttacgtgggattgtgaaagagcaactggcagctgtgacccaaaaatgggatagc
ctaacagggcaattgagtgacagatgtgactggattgaccaagccattgttaaaagcaca
cagtatcaaagcctgctgagaagcctttctgataaactgagtgacttggataataaactc
agcagcagtctggctgtgagcacgcaccctgatgctatgaaccaacagttggaaacagcc
caaaaaatgaagcaggagatacagcaggaaaagaagcagataaaagtggcccaggcactc
tgtgaggatttgtcagcactggttaaagaagagtacttgaaagcagaacttagtaggcaa
ctagaaggcatcttaaaatcatttaaggatgttgaacagaaagcagagaatcatgtccag
caccttcagtcggcctgtgcaagctctcatcaatttcagcaaatgtctagagattttcag
gcttggctggatacaaagaaagaagagcaaaacaaatctcatccaatatctgccaaactc
gatgtcttggagtcattaattaaagatcataaagactttagtaaaactttgaccgctcag
tctcatatgtatgaaaaaaccattgcagaaggtgaaaatctgttattaaaaacacaaggg
tctgagaaggcagccttacagttacagcttaatacaattaaaaccaattgggatacattt
aataagcaggtgaaagaaagagaaaacaagttaaaagagtcattggaaaaagcccttaag
tataaagagcaagtagagactctctggccatggatagacaaatgccaaaacaacctggag
gaaataaaattttgcttggatcctgctgaaggagagaattctattgccaagttaaagtct
ctgcagaaggaaatggaccaacactttggtatggtagaattactgaacaacacagccaat
agcttgctcagtgtctgtgagatagataaagaagttgttacagatgagaataagtcactg
atccagaaggtggacatggtcactgaacaacttcacagtaagaaattctgtctggagaac
atgactcagaagtttaaagaatttcaagaagtttccaaagaatctaaaaggcagcttcag
tgtgcaaaggagcagctagatatccatgattcgctgggatcccaggcttacagtaacaaa
tacctgaccatgttgcaaactcagcagaaatcacttcaggccttgaagcatcaggtagat
ttggctaaaagacttgcacaggaccttgtggtagaggcctcagactcaaagggaacctct
gatgttttattacaagtggaaaccatagctcaagagcatagtacactaagtcagcaggtt
gatgaaaagtgttctttcttagaaaccaagcttcagggcattgggcatttccagaatacc
attcgagaaatgttttctcagttcgcagagtttgatgatgaactggatagcatggctcca
gtggggagagatgcagaaacattgcaaaagcaaaaggaaactataaaagcctttctaaag
aaactagaagccctcatggcaagcaatgacaatgccaataaaacctgcaagatgatgtta
gccacagaagaaacctctcctgaccttgttggaatcaaaagggacttggaggccttaagc
aaacaatgcaacaagttactggaccgagcccaagccagagaagagcaggttgaagggaca
attaagcgccttgaagaattttacagcaaattgaaagaattttctattctgctccagaaa
gccgaagaacatgaagagtcacaaggtcctgttggtatggaaacggagacaattaatcag
cagcttaacatgttcaaggtattccagaaagaagagattgaacccttgcaaggtaaacag
caagatgtaaactggttaggtcaaggccttattcagagtgctgccaaaagcactagcact
cagggcttggagcatgacctggatgatgtcaatgcacggtggaagactctcaataagaag
gtggctcagcgagcagcccagctgcaggaggccttgctgcactgtgggaggttccaggat
gccctggagtccctgctcagctggatggtggacactgaggagcttgtggccaatcagaag
cccccgtcggctgagttcaaagtggtaaaggcccagatacaagaacaaaagcttctccag
agattgttggatgaccgaaaatctacggtggaggtaatcaaacgagaaggagaaaaaatt
gctacaacagcagagcccgcagataaagtgaagattttgaaacagctcagtctcttggat
agcagatgggaggcattgcttaataaagctgaaacaaggaatcgtcagttggaaggtatc
tcggtggtagcacagcaatttcatgaaaccttagaaccactgaacgagtggcttacaacc
atagaaaagaggctggtgaattgtgaacccataggaacccaagcatctaaacttgaggaa
caaattgcacagcacaaagttctgcaagaggacatcttactcaggaaacaaaatgtagat
caggctttactaaatggtttagaactacttaaacaaaccacaggtgatgaagttttaata
attcaagataaattggaagccattaaagcaaggtacaaagacattactaaactgagcact
gatgtggccaagactctggaacaggcgctgcagcttgcaaggcggctgcactccacacac
gaagagctgtgtacctggctggacaaagtggaggtggaattactttcatatgaaactcag
gttctgaaaggagaagaagcaagtcaagcacaaatgagaccaaaggaactgaaaaaggaa
gctaagaacaacaaagccttactggactcccttaatgaagtgagcagtgctttgctggaa
ctggtaccatggagggcaagagaaggacttgagaaaatggtagctgaggacaatgagcgc
taccgattagtgagcgacaccatcactcagaaggtggaggagatcgatgcagccattctg
cgatcacagcagtttgaccaagcagctgatgctgagttatcctggattactgaaacagaa
aaaaaattgatgtctctgggtgacatcaggcttgagcaagaccagacttctgctcagctt
caagttcaaaagacattcaccatggagattttgagacacaaggatattattgatgacctt
gttaaatctgggcataaaatcatgaccgcatgcagtgaagaggaaaagcaatcaatgaag
aaaaaactggacaaggtactgaagaactatgataccatctgccagattaattcagaaagg
tatctgcagctggaacgggcacagtccctggttaaccaattctgggaaacatatgaagaa
ctttggccatggctgacagaaacacaatcaatcatctctcagcttcccgccccagccctt
gaatatgaaactctaaggcagcagcaggaagaacatcggcaactgcgtgagttgatagct
gaacacaagcctcatatagataagatgaacaaaactgggccacagttactggaattgagc
cctggggaaggcttttctatccaagagaagtatgtggcagccgacaccctttacagtcaa
attaaagaagatgtcaaaaagcgtgctgtggcactggatgaagccatttctcaatcaact
cagttccatgacaagatagatcagatccttgagagcctggaacgcatcgtggaacgtctg
aggcagccaccctctatctctgcagaggttgagaagatcaaggaacagatcagtgaaaat
aagaatgtgtcagtagacatggaaaagctacagccgttgtatgaaactcttaaacagagg
ggagaggaaatgattgctagatctggggggactgataaagacatatctgccaaagctgtt
caggataagcttgaccaaatggttttcatttgggagaacatacacacactggtggaagag
agggaagccaaactactggatgtgatggagctagcagaaaagttctggtgtgatcacatg
tcattgatagttaccattaaagatactcaagatttcatccgggacctggaagatcctgga
attgatccttcagtagtaaaacaacagcaagaagcagcagagaccataagggaagaaata
gatggactacaggaggagctggatatagttattaacctaggttctgaactcattgcggca
tgtggggagcctgataaacccattgtcaagaagagtatagatgagttaaattcagcatgg
gattctctaaataaagcttggaaagaccggattgacaaacttgaggaggcaatgcaggct
gccgttcagtaccaggatggactgcaggcggtatttgactgggtagatattgcaggtggt
aaattagcttcaatgtctccaattggaacagatctcgaaactgtcaagcagcagattgaa
gagctaaagcaatttaagtctgaggcctatcaacagcagatagaaatggaaagactgaat
catcaagcagagcttttgctaaagaaagtaacagaagagagtgacaaacacactgttcaa
gacccattaatggaactgaaattgatatgggatagcctggaggagagaatcatcaacaga
cagcataaactggagggtgctctattagccttgggtcagttccaacatgccctggatgag
ctcctggcatggctgacacacaccgagggcttgctaagtgagcagaaacctgttggagga
gaccctaaagccattgaaattgaacttgccaagcatcatgtgctccaaaatgatgtatta
gcccatcagtccacagtggaagccgttaataaagcaggaaatgatctaattgaatcaagt
gcaggagaagaagcaagcaaccttcagaacaagctagaggttttaaatcaacgctggcaa
aatgttttggaaaaaacagaacaaaggaagcagcagctggatggtgccttgcgccaggcc
aaagggttccatggcgaaattgaggatttgcagcagtggctgactgacacggagcgtcat
ctgttggcatctaaaccgctgggaggtttaccggaaacagccaaggagcagcttaatgtc
catatggaagtctgtgctgcctttgaagctaaagaagaaacatataagagtctgatgcag
aaaggccagcagatgcttgcaagatgcccaaaatctgcagagacaaatattgaccaagac
ataaataacttgaaagaaaaatgggaatcggtggaaaccaaactcaatgaaaggaaaact
aaactggaagaggctctcaacttggcaatggagttccacaattctctccaagacttcatc
aactggcttactcaggctgaacagaccctaaatgtagcttctcggccaagtctcatcttg
gacacagtcttatttcaaattgacgaacacaaggtttttgccaatgaagtaaattctcat
cgtgagcagataatagagctggacaaaactggaacccacctaaaatattttagtcagaaa
caagatgttgttctaatcaagaatctacttatcagtgtacaaagtcgatgggaaaaagtg
gttcaacggttggtagagagaggaagatctttggatgatgcaaggaagagagccaagcag
ttccatgaagcttggagtaaacttatggagtggctagaagagtcagaaaagtctttggat
tctgaactggaaatcgcaaatgatccagacaaaataaaaacacaacttgcacaacataag
gagtttcagaaatcactcggagccaagcattctgtctacgacaccaccaacaggactgga
cgttctctgaaggagaaaacctccctggctgatgacaacctgaaactggatgacatgctg
agtgaactcagagacaaatgggataccatatgtggaaaatctgtggaaagacaaaacaaa
ttggaggaagccctgttattttctggacaattcacagatgccctacaggctctcattgat
tggttatatagagttgaaccccagctggcagaagaccagcctgttcatggagacattgat
ttggtgatgaatctgatcgataatcacaaggccttccaaaaagagttggggaagaggacc
agcagtgtgcaggccctgaagcgctcagcccgagaactcatagaaggcagtcgggatgac
tcctcctgggtcaaggtccagatgcaggaattaagcacacgctgggagaccgtgtgtgca
ctttctatatcaaagcaaacacggttagaagcagccctgcgtcaggcagaggaattccac
tcggtggtacatgccctcttggagtggctggctgaggcggagcaaaccctgcgtttccat
ggtgtcctcccagatgatgaggatgctctccggactctcattgatcagcataaagaattc
atgaagaaactggaagaaaagagagctgaactaaataaagccaccactatgggcgacacc
gttttggctatctgccaccccgactccatcactaccattaagcactggataacaatcatc
cgggcgaggtttgaggaggtgctggcctgggcaaagcaacatcagcagagattagcaagt
gctctggctgggcttattgccaaacaggaattgttggaagctttgctggcttggttgcaa
tgggctgaaactacacttactgataaggataaagaagtcatcccccaggagatcgaagag
gtgaaagcactcattgcagaacaccagaccttcatggaggaaatgaccagaaaacagcct
gatgttgataaagtaacgaagacctataagaggagagctgctgatccttcctcattacaa
tcccatattccagtcttggataagggacgagcaggaagaaaacgctttccagcatcaagc
ttgtatccctctgggtcacagacacaaattgaaaccaaaaatcctagggtaaacttactg
gtgagcaaatggcagcaagtctggctcctggcgttggaaagaaggaggaaactcaatgat
gccttggacagactagaggagctgagggaatttgctaactttgattttgatatttggcgc
aaaaaatacatgcgatggatgaatcacaagaaatctcgagtgatggacttcttcaggaga
attgataaagaccaggatgggaaaataacgcggcaggaatttattgatggaattctttcc
tcaaagtttccaaccagtcgcttggagatgagcgcagttgcagacatctttgacagagat
ggcgatggatatattgactactatgaatttgtagcagcccttcacccaaataaagatgca
tataaacctatcacagatgccgacaaaatcgaagatgaggtgacaaggcaggtagctaag
tgtaaatgtgcaaagcgatttcaagttgagcagattggtgataataaatacaggttcttc
ctgggaaatcagtttggagactcccagcaactgcgactggtccggatcctgcggagtact
gtgatggttcgtgttggaggtggatggatggcacttgatgagttcttagtgaaaaatgat
ccttgcagggccaaaggaaggacaaacatggaactgcgtgagaagttcattttagcagat
ggtgccagccagggtatggctgctttccgaccccgaggccgaagatcccggccatcatca
cgaggcgcttcacccaacagatccacttctgtgtccagtcaggctgcgcaggcggcctcc
ccacaggtccctgccaccaccacacccaagattctccatcctttaacacgcaattatggt
aaaccatggttgacaaacagcaaaatgtcaactccttgtaaagcagcagagtgctcagac
tttcccgtgccatctgcagagggaacgccaatacaaggaagcaagcttcgacttccagga
tatttatcagggaaaggcttccactctggggaggacagtggcttgataacaactgcagct
gccagagtccgaacacagtttgctgattccaagaagactcccagccgaccaggaagtcga
gctggaagcaaagctggcagcagggccagcagccgccgaggcagtgatgcatcagacttt
gacatttcagaaatccagtccgtgtgctcagatgtggaaactgtcccccagacacacaga
cctacaccccgagcaggttctcggccatccacagcgaagccttcaaaaatccccacgccc
cagaggaaatcacctgccagcaaattggacaagtcctcaaagagatagtgcaattggttc
taccaaggcccttccttgagcatttattatttaagtttgaacgatgtaaaatatggtgta
gaaattcttgtgaaatattgcaagaggcgagtttaaaattctgcagatggccttatttgt
gtatttgtctttttattttatctgtataattttttttgtcagatattctggggttaaagt
cacatcatatgtgaggaggaaaagtttaacatgaactaacatttctgcactgtaacgtgc
cgggcacacactaaactcagttactgtacctacaggtaagtctacatcctctctgacagc
cacagcactacatcaatccctgacgttagggatacctcatgacattttcctgtttttatg
gaaactctgagaagctgaatgatacatgcaggggatattttttgagatgatttaaatgta
aaccaaaagatggaagacaaaaagacaaacacacccacacgcagtctttgcagtatctga
cagagaactcacaggaagttacttcaagcacttgccagtactatgatattcaagtacctt
gcagcatttctctgccattgctttcaatgaggccagaggcatcctggatattagacctat
tatactgtaagaatataagtataaagtgcgttcatatacatgtgaggttttcttttgctt
gagtggacagtagcacctgtatcattgaactcattttgtatcagagcaattttgcttgca
gaaagctatgaaataaaacacgtcccttaactgc

TABLE 14
Protein Sequence of PN9109

WLVEKELMVSVLGPLSIDPNMLNTQRQQVQILLQEFATRKPQYEQLTAAGQGILSRPGED
PSLRGIVKEQLAAVTQKWDSLTGQLSDRCDWIDQAIVKSTQYQSLLRSLSDKLSDLDNKL
SSSLAVSTHPDAMNQQLETAQKMKQEIQQEKKQIKVAQALCEDLSALVKEEYLKAELSRQ
LEGILKSFKDVEQKAENHVQHLQSACASSHQFQQMSRDFQAWLDTKKEEQNKSHPISAKL
DVLESLIKDHKDFSKTLTAQSHMYEKTIAEGENLLLKTQGSEKAALQLQLNTIKTNWDTF
NKQVKERENKLKESLEKALKYKEQVETLWPWIDKCQNNLEEIKFCLDPAEGENSIAKLKS
LQKEMDQHFGMVELLNNTANSLLSVCEIDKEVVTDENKSLIQKVDMVTEQLHSKKFCLEN
MTQKFKEFQEVSKESKRQLQCAKEQLDIHDSLGSQAYSNKYLTMLQTQQKSLQALKHQVD
LAKRLAQDLVVEASDSKGTSDVLLQVETIAQEHSTLSQQVDEKCSFLETKLQGIGHFQNT
IREMFSQFAEFDDELDSMAPVGRDAETLQKQKETIKAFLKKLEALMASNDNANKTCKMML
ATEETSPDLVGIKRDLEALSKQCNKLLDRAQAREEQVEGTIKRLEEFYSKLKEFSILLQK
AEEHEESQGPVGMETETINQQLNMFKVFQKEEIEPLQGKQQDVNWLGQGLIQSAAKSTST
QGLEHDLDDVNARWKTLNKKVAQRAAQLQEALLHCGRFQDALESLLSWMVDTEELVANQK
PPSAEFKVVKAQIQEQKLLQRLLDDRKSTVEVIKREGEKIATTAEPADKVKILKQLSLLD
SRWEALLNKAETRNRQLEGISVVAQQFHETLEPLNEWLTTIEKRLVNCEPIGTQASKLEE
QIAQHKVLQEDILLRKQNVDQALLNGLELLKQTTGDEVLIIQDKLEAIKARYKDITKLST
DVAKTLEQALQLARRLHSTHEELCTWLDKVEVELLSYETQVLKGEEASQAQMRPKELKKE
AKNNKALLDSLNEVSSALLELVPWRAREGLEKMVAEDNERYRLVSDTITQKVEEIDAAIL
RSQQFDQAADAELSWITETEKKLMSLGDIRLEQDQTSAQLQVQKTFTMEILRHKDIIDDL
VKSGHKIMTACSEEEKQSMKKKLDKVLKNYDTICQINSERYLQLERAQSLVNQFWETYEE
LWPWLTETQSIISQLPAPALEYETLRQQQEEHRQLRELIAEHKPHIDKMNKTGPQLLELS
PGEGFSIQEKYVAADTLYSQIKEDVKKRAVALDEAISQSTQFHDKIDQILESLERIVERL
RQPPSISAEVEKIKEQISENKNVSVDMEKLQPLYETLKQRGEEMIARSGGTDKDISAKAV
QDKLDQMVFIWENIHTLVEEREAKLLDVMELAEKFWCDHMSLIVTIKDTQDFIRDLEDPG
IDPSVVKQQQEAAETIREEIDGLQEELDIVINLGSELIAACGEPDKPIVKKSIDELNSAW
DSLNKAWKDRIDKLEEAMQAAVQYQDGLQAVFDWVDIAGGKLASMSPIGTDLETVKQQIE
ELKQFKSEAYQQQIEMERLNHQAELLLKKVTEESDKHTVQDPLMELKLIWDSLEERIINR
QHKLEGALLALGQFQHALDELLAWLTHTEGLLSEQKPVGGDPKAIEIELAKHHVLQNDVL
AHQSTVEAVNKAGNDLIESSAGEEASNLQNKLEVLNQRWQNVLEKTEQRKQQLDGALRQA
KGFHGEIEDLQQWLTDTERHLLASKPLGGLPETAKEQLNVHMEVCAAFEAKEETYKSLMQ
KGQQMLARCPKSAETNIDQDINNLKEKWESVETKLNERKTKLEEALNLAMEFHNSLQDFI
NWLTQAEQTLNVASRPSLILDTVLFQIDEHKVFANEVNSHREQIIELDKTGTHLKYFSQK
QDVVLIKNLLISVQSRWEKVVQRLVERGRSLDDARKRAKQFHEAWSKLMEWLEESEKSLD
SELEIANDPDKIKTQLAQHKEFQKSLGAKHSVYDTTNRTGRSLKEKTSLADDNLKLDDML
SELRDKWDTICGKSVERQNKLEEALLFSGQFTDALQALIDWLYRVEPQLAEDQPVHGDID
LVMNLIDNHKAFQKELGKRTSSVQALKRSARELIEGSRDDSSWVKVQMQELSTRWETVCA
LSISKQTRLEAALRQAEEFHSVVHALLEWLAEAEQTLRFHGVLPDDEDALRTLIDQHKEF
MKKLEEKRAELNKATTMGDTVLAICHPDSITTIKHWITIIRARFEEVLAWAKQHQQRLAS
ALAGLIAKQELLEALLAWLQWAETTLTDKDKEVIPQEIEEVKALIAEHQTFMEEMTRKQP
DVDKVTKTYKRRAADPSSLQSHIPVLDKGRAGRKRFPASSLYPSGSQTQIETKNPRVNLL
VSKWQQVWLLALERRRKLNDALDRLEELREFANFDFDIWRKKYMRWMNHKKSRVMDFFRR
IDKDQDGKITRQEFIDGILSSKFPTSRLEMSAVADIFDRDGDGYIDYYEFVAALHPNKDA
YKPITDADKIEDEVTRQVAKCKCAKRFQVEQIGDNKYRFFLGNQFGDSQQLRLVRILRST
VMVRVGGGWMALDEFLVKNDPCRAKGRTNMELREKFILADGASQGMAAFRPRGRRSRPSS
RGASPNRSTSVSSQAAQAASPQVPATTTPKILHPLTRNYGKPWLTNSKMSTPCKAAECSD
FPVPSAEGTPIQGSKLRLPGYLSGKGFHSGEDSGLITTAAARVRTQFADSKKTPSRPGSR
AGSKAGSRASSRRGSDASDFDISEIQSVCSDVETVPQTHRPTPRAGSRPSTAKPSKIPTP
QRKSPASKLDKSSKR

### Example 13

### Identification of C-NAP 1/KIAA1106 Interaction

A yeast two-hybrid system as described in Example 1 using amino acids encoded by nucleotides 4419-5336 of C-NAP1 (GB accession no. AF049105) as bait was performed. One clone that was identified by this procedure included amino acids encoded by nucleotides 2366-2985 of KIAA1106 (GB accession no. AB029029).

### Example 14

### Generation of Polyclonal Antibody Against Protein Complexes

As shown above, MAPKAP-K3 interacts with AP-3 delta to form a complex. A complex of the two proteins is prepared, e.g., by mixing purified preparations of each of the two proteins. If desired, the protein complex can be stabilized by cross-linking the proteins in the complex, by methods known to those of skill in the art. The protein complex is used to immunize rabbits and mice using a procedure similar to that described by Harlow et al. (1988). This procedure has been shown to generate Abs against various other proteins (for example, see Kraemer et al., 1993).

Briefly, purified protein complex is used as immunogen in rabbits. Rabbits are immunized with 100 μg of the protein in complete Freund's adjuvant and boosted twice in three-week intervals, first with 100 μg of immunogen in incomplete Freund's adjuvant, and followed by 100 μg of immunogen in PBS. Antibody-containing serum is collected two weeks thereafter. The antisera is preadsorbed with MAPKAP-K3 and AP-3 delta, such that the remaining antisera comprises antibodies which bind conformational epitopes, i.e., complex-specific epitopes, present on the MAPKAP-K3/AP-3 delta complex but not on the monomers.

Polyclonal antibodies against each of the complexes set forth in Tables 1-12 are prepared in a similar manner by mixing the specified proteins together, immunizing an animal and isolating antibodies specific for the protein complex, but not for the individual proteins.

Polyclonal antibodies against the protein set forth in Table 14 are prepared in a similar manner by immunizing an animal with the protein and isolating antibodies specific for the protein.

### Example 15

### Generation of Monoclonal Antibodies Specific for Protein Complexes

Monoclonal antibodies are generated according to the following protocol. Mice are immunized with immunogen comprising MAPKAP-K3/AP-3 delta complexes conjugated to keyhole limpet hemocyanin using glutaraldehyde or EDC as is well known in the art. The complexes can be prepared as described in Example 14, and may also be stabilized by cross-linking. The immunogen is mixed with an adjuvant. Each mouse receives four injections of 10 to 100 μg of immunogen, and after the fourth injection blood samples are taken from the mice to determine if the serum contains antibody to the immunogen. Serum titer is determined by ELISA or RIA. Mice with sera indicating the presence of antibody to the immunogen are selected for hybridoma production.

Spleens are removed from immune mice and a single-cell suspension is prepared (Harlow et al., 1988). Cell fusions are performed essentially as described by Kohler et al. (1975). Briefly, P3.65.3 myeloma cells (American Type Culture Collection, Rockville, Md.) or NS-1 myeloma cells are fused with immune spleen cells using polyethylene glycol as described by Harlow et al. (1988). Cells are plated at a density of 2×10⁵ cells/well in 96-well tissue culture plates. Individual wells are examined for growth, and the supernatants of wells with growth are tested for the presence of MAPKAP-K3/AP-3 delta complex-specific antibodies by ELISA or RIA using MAPKAP-K3/AP-3 delta complex as target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to MAPKAP-K3 alone or to AP-3 delta alone, to determine which are specific for the MAPKAP-K3/AP-3 delta complex as opposed to those that bind to the individual proteins.

Monoclonal antibodies against each of the complexes set forth in Tables 1-12 are prepared in a similar manner by mixing the specified proteins together, immunizing an animal, fusing spleen cells with myeloma cells and isolating clones which produce antibodies specific for the protein complex, but not for the individual proteins.

Monoclonal antibodies against the protein set forth in Table 14 are prepared in a similar manner by immunizing an animal with the protein, fusing spleen cells with myeloma cells and isolating clones which produce antibodies specific for the protein.

### Example 16

### In vitro Identification of Modulators for Protein-protein Interactions

The present invention is useful in screening for agents that modulate the interaction of MAPKAP-K3 and AP-3 delta. The knowledge that MAPKAP-K3 and AP-3 delta form a complex is useful in designing such assays. Candidate agents are screened by mixing MAPKAP-K3 and AP-3 delta (a) in the presence of a candidate agent, and (b) in the absence of the candidate agent. The amount of complex formed is measured for each sample. An agent modulates the interaction of MAPKAP-K3 and AP-3 delta if the amount of complex formed in the presence of the agent is greater than (promoting the interaction), or less than (inhibiting the interaction) the amount of complex formed in the absence of the agent. The amount of complex is measured by a binding assay, which shows the formation of the complex, or by using antibodies immunoreactive to the complex.

Briefly, a binding assay is performed in which immobilized MAPKAP-K3 is used to bind labeled AP-3 delta. The labeled AP-3 delta is contacted with the immobilized MAPKAP-K3 under aqueous conditions that permit specific binding of the two proteins to form an MAPKAP-K3/AP-3 delta complex in the absence of an added test agent. Particular aqueous conditions may be selected according to conventional methods. Any reaction condition can be used as long as specific binding of MAPKAP-K3/AP-3 delta occurs in the control reaction. A parallel binding assay is performed in which the test agent is added to the reaction mixture. The amount of labeled AP-3 delta bound to the immobilized MAPKAP-K3 is determined for the reactions in the absence or presence of the test agent. If the amount of bound, labeled AP-3 delta in the presence of the test agent is different than the amount of bound labeled AP-3 delta in the absence of the test agent, the test agent is a modulator of the interaction of MAPKAP-K3 and AP-3 delta.

Candidate agents for modulating the interaction of each of the protein complexes set forth in Tables 1-12 are screened in vitro in a similar manner.

### Example 17

### In vivo Identification of Modulators for Protein-protein Interactions

In addition to the in vitro method described in Example 16, an in vivo assay can also be used to screen for agents which modulate the interaction of MAPKAP-K3 and AP-3 delta. Briefly, a yeast two-hybrid system is used in which the yeast cells express (1) a first fusion protein comprising MAPKAP-K3 or a fragment thereof and a first transcriptional regulatory protein sequence, e.g., GAL4 activation domain, (2) a second fusion protein comprising AP-3 delta or a fragment thereof and a second transcriptional regulatory protein sequence, e.g., GAL4 DNA-binding domain, and (3) a reporter gene, e.g., β-galactosidase, which is transcribed when an intermolecular complex comprising the first fusion protein and the second fusion protein is formed. Parallel reactions are performed in the absence of a test agent as the control and in the presence of the test agent. A functional MAPKAP-K3/AP-3 delta complex is detected by detecting the amount of reporter gene expressed. If the amount of reporter gene expression in the presence of the test agent is different than the amount of reporter gene expression in the absence of the test agent, the test agent is a modulator of the interaction of MAPKAP-K3 and AP-3 delta.

Candidate agents for modulating the interaction of each of the protein complexes set forth in Tables 1-12 are screened in vivo in a similar manner.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### BIBLIOGRAPHY

Altschul, S. F. et al. (1990). Basic local alignment search tool. J. Mol. Biol. 215:403-410.

Altschul, S. F. et al. (1997). Gapped BLAST and PSI-BLAST: a new generation of protein database search programs. Nucl. Acids Res. 25:3389-3402.

Aronheim et al., 1997. Isolation of an AP-1 repressor by a novel method for detecting protein-protein interactions. Mol. Cell. Biol. 17:3094-3102.

Bartel, P. L. et al. (1993). “Using the 2-hybrid system to detect protein-protein interactions.” In: Cellular Interactions in Development: A Practical Approach, Oxford University Press, pp. 153-179.

Bartel, P. L. et al. (1996). A protein linkage map of Escherichia coli bacteriophage T7. Nat Genet 12:72-77.

Bartel, P. L. and Fields, S. (1997). The Yeast Two-Hybrid System. New York: Oxford University Press.

Bieniasz, P. D. et al. (1998). Recruitment of a protein complex containing Tat and cyclin T1 to TAR governs the species specificity of HIV-1 Tat. EMBO J. 17:7056-65.

Best, J. L. et al. (1995). Cloning of a full-length cDNA sequence encoding a cdc2-related protein kinase from human endothelial cells. Biochem Biophys Res Commun. 208:562-8.

Carter, D. A. (1997). Modulation of cellular AP-1 DNA binding activity by heat shock proteins. FEBS Lett. 416:81-5.

Cheng, M. et al. (1996). ERK3 is a constitutively nuclear protein kinase. J. Biol. Chem. 271:8951-8.

Chevray, P. M. and Nathans, D. N. (1992). Protein interaction cloning in yeast: identification of mammalian proteins that interact with the leucine zipper of Jun. Proc. Natl. Acad. Sci. USA 89:5789-5793.

Clifton, A. D. et al. (1996). A comparison of the substrate specificity of MAPKAP kinase-2 and MAPKAP kinase-3 and their activation by cytokines and cellular stress. FEBS Lett. 392:209-14.

Colwill, K. et al. (1996). The Clk/Sty protein kinase phosphorylates SR splicing factors and regulates their intranuclear distribution. EMBO J. 15:265-75.

Fields, S. and Song, O- K. (1989). A novel genetic system to detect protein-protein interactions. Nature 340:245-246.

Fry, A. M. et al. (1998). C-Nap1, a novel centrosomal coiled-coil protein and candidate substrate of the cell cycle-regulated protein kinase Nek2. J Cell Biol. 141:1563-74.

Fu, T. J. et al., (1999). Cyclin K functions as a CDK9 regulatory subunit and participates in RNA polymerase II transcription. J Biol. Chem. 274:34527-30.

What is claimed is:

1. An isolated protein complex comprising two proteins, the protein complex selected from the group consisting of
(a) a complex set forth in Table 1;
(b) a complex set forth in Table 2;
(c) a complex set forth in Table 3;
(d) a complex set forth in Table 4;
(e) a complex set forth in Table 5;
(f) a complex set forth in Table 6;
(g) a complex set forth in Table 7;
(h) a complex set forth in Table 8;
(i) a complex set forth in Table 9;
(h) a complex set forth in Table 10;
(k) a complex set forth in Table 11; and
(l) a complex set forth in Table 12.

2. The protein complex of claim 1, wherein said protein complex comprises complete proteins.

3. The protein complex of claim 1, wherein said protein complex comprises a fragment of one protein and a complete protein of anther protein.

4. The protein complex of claim 1, wherein said protein complex comprises fragments of proteins.

5. An isolated antibody selectively immunoreactive with the protein complex of claim 1

6. The antibody of claim 5, wherein said antibody is a monoclonal antibody.

7. A method for diagnosing a physiological disorder in an animal, which comprises assaying for:
(a) whether a protein complex set forth in any one of Tables 1-12 is present in a tissue extract;
(b) the ability of proteins to form a protein complex set forth in any one of Tables 1-12; and
(c) a mutation in a gene encoding a protein of a protein complex set forth in any one of Tables 1-12.

8. The method of claim 7, wherein said animal is a human.

9. The method of claim 7, wherein the diagnosis is for a predisposition to said physiological disorder.

10. The method of claim 7, wherein the diagnosis is for the existence of said physiological disorder.

11. The method of claim 7, wherein said assay comprises a yeast two-hybrid assay.

12. The method of claim 7, wherein said assay comprises measuring in vitro a complex formed by combining the proteins of the protein complex, said proteins isolated from said animal.

13. The method of claim 12, wherein said complex is measured by binding with an antibody specific for said complex.

14. The method of claim 7, wherein said assay comprises mixing an antibody specific for said protein complex with a tissue extract from said animal and measuring the binding of said antibody.

15. A method for determining whether a mutation in a gene encoding one of the proteins of a protein complex set forth in any one of Tables 1-12 is useful for diagnosing a physiological disorder, which comprises assaying for the ability of said protein with said mutation to form a complex with the other protein of said protein complex, wherein an inability to form said complex is indicative of said mutation being useful for diagnosing a physiological disorder.

16. The method of claim 15, wherein said gene is an animal gene.

17. The method of claim 16, wherein said animal is a human.

18. The method of claim 15, wherein the diagnosis is for a predisposition to a physiological disorder.

19. The method of claim 15, wherein the diagnosis is for the existence of a physiological disorder.

20. The method of claim 15, wherein said assay comprises a yeast two-hybrid assay.

21. The method of claim 15, wherein said assay comprises measuring in vitro a complex formed by combining the proteins of the protein complex, said proteins isolated from an animal.

22. The method of claim 21, wherein said animal is a human.

23. The method of claim 21, wherein said complex is measured by binding with an antibody specific for said complex.

24. A method for screening for drug candidates capable of modulating the interaction of the proteins of a protein complex set forth in any one of Tables 1-12, which comprises:
(a) combining the proteins of said protein complex in the presence of a drug to form a first complex;
(b) combining the proteins in the absence of said drug to form a second complex;
(c) measuring the amount of said first complex and said second complex; and
(d) comparing the amount of said first complex with the amount of said second complex,
wherein if the amount of said first complex is greater than, or less than the amount of said second complex, then the drug is a drug candidate for modulating the interaction of the proteins of said protein complex.

25. The method of claim 24, wherein said screening is an in vitro screening.

26. The method of claim 24, wherein said complex is measured by binding with an antibody specific for said protein complexes.

27. The method of claim 24, wherein if the amount of said first complex is greater than the amount of said second complex, then said drug is a drug candidate for promoting the interaction of said proteins.

28. The method of claim 24, wherein if the amount of said first complex is less than the amount of said second complex, then said drug is a drug candidate for inhibiting the interaction of said proteins.

29. A non-human animal model for a physiological disorder wherein the genome of said animal or an ancestor thereof has been modified such that the formation of a protein complex set forth in any one of Tables 1-12 has been altered.

30. The non-human animal model of claim 29, wherein the formation of said protein complex has been altered as a result of:
(a) over-expression of at least one of the proteins of said protein complex;
(b) replacement of a gene for at least one of the proteins of said protein complex with a gene from a second animal and expression of said protein;
(c) expression of a mutant form of at least one of the proteins of said protein complex;
(d) a lack of expression of at least one of the proteins of said protein complex; or
(e) reduced expression of at least one of the proteins of said protein complex.

31. A cell line obtained from the animal model of claim 29.

32. A non-human animal model for a physiological disorder, wherein the biological activity of a protein complex set forth in any one of Tables 1-12 has been altered.

33. The non-human animal model of claim 32, wherein said biological activity has been altered as a result of:
(a) disrupting the formation of said complex; or
(b) disrupting the action of said complex.

34. The non-human animal model of claim 32, wherein the formation of said complex is disrupted by binding an antibody to at least one of the proteins which form said protein complex.

35. The non-human animal model of claim 32, wherein the action of said complex is disrupted by binding an antibody to said complex.

36. The non-human animal model of claim 32, wherein the formation of said complex is disrupted by binding a small molecule to at least one of the proteins which form said protein complex.

37. The non-human animal model of claim 32, wherein the action of said complex is disrupted by binding a small molecule to said complex.

38. A cell in which the genome of cells of said cell line has been modified to produce at least one protein complex set forth in any one of Tables 1-12.

39. A cell line in which the genome of the cells of said cell line has been modified to eliminate at least one protein of a protein complex set forth in any one of Tables 1-12.

40. A method of screening for drug candidates useful in treating a physiological disorder which comprises the steps of:
(a) measuring the activity of a protein selected from the proteins set forth in Tables 1-12 in the presence of a drug,
(b) measuring the activity of said protein in the absence of said drug, and
(c) comparing the activity measured in steps (1) and (2),
wherein if there is a difference in activity, then said drug is a drug candidate for treating said physiological disorder.

41. An isolated DNA molecule comprising a nucleotide sequence coding for the amino acid sequence set forth in Table 14.

42. The isolated DNA molecule of claim 41, wherein said nucleotide sequence comprises the nucleotide sequence set forth in Table 13.

43. An isolated protein comprising an amino acid sequence set forth in Table 14.


SEQUENCE LISTING (4 sequences)

SEQ ID NO: 1 - DNA, 40 bp, Artificial Sequence
  primer for yeast two-hybrid
gcaggaaacagctatgaccatacagtcagcggccgccacc

SEQ ID NO: 2 - DNA, 39 bp, Artificial Sequence
  primer for yeast two hybrid
acggccagtcgcgtggagtgttatgtcatgcggccgcta

SEQ ID NO: 3 - DNA, 9274 bp, Homo sapiens
tggcttgtggaaaaagaacttatggtcagtgttcttgggcccttgtcaattgacccaaatatgctaaacacacaaaggcagcaggtgcagattttgctgcaagaattcgccactcggaaacctcaatatgaacagctgacagcagctggtcagggcattctgagcaggcctggagaagacccttctttacgtgggattgtgaaagagcaactggcagctgtgacccaaaaatgggatagcctaacagggcaattgagtgacagatgtgactggattgaccaagccattgttaaaagcacacagtatcaaagcctgctgagaagcctttctgataaactgagtgacttggataataaactcagcagcagtctggctgtgagcacgcaccctgatgctatgaaccaacagttggaaacagcccaaaaaatgaagcaggagatacagcaggaaaagaagcagataaaagtggcccaggcactctgtgaggatttgtcagcactggttaaagaagagtacttgaaagcagaacttagtaggcaactagaaggcatcttaaaatcatttaaggatgttgaacagaaagcagagaatcatgtccagcaccttcagtcggcctgtgcaagctctcatcaatttcagcaaatgtctagagattttcaggcttggctggatacaaagaaagaagagcaaaacaaatctcatccaatatctgccaaactcgatgtcttggagtcattaattaaagatcataaagactttagtaaaactttgaccgctcagtctcatatgtatgaaaaaaccattgcagaaggtgaaaatctgttattaaaaacacaagggtctgagaaggcagccttacagttacagcttaatacaattaaaaccaattgggatacatttaataagcaggtgaaagaaagagaaaacaagttaaaagagtcattggaaaaagcccttaagtataaagagcaagtagagactctctggccatggatagacaaatgccaaaacaacctggaggaaataaaattttgcttggatcctgctgaaggagagaattctattgccaagttaaagtctctgcagaaggaaatggaccaacactttggtatggtagaattactgaacaacacagccaatagcttgctcagtgtctgtgagatagataaagaagttgttacagatgagaataagtcactgatccagaaggtggacatggtcactgaacaacttcacagtaagaaattctgtctggagaacatgactcagaagtttaaagaatttcaagaagtttccaaagaatctaaaaggcagcttcagtgtgcaaaggagcagctagatatccatgattcgctgggatcccaggcttacagtaacaaatacctgaccatgttgcaaactcagcagaaatcacttcaggccttgaagcatcaggtagatttggctaaaagacttgcacaggaccttgtggtagaggcctcagactcaaagggaacctctgatgttttattacaagtggaaaccatagctcaagagcatagtacactaagtcagcaggttgatgaaaagtgttctttcttagaaaccaagcttcagggcattgggcatttccagaataccattcgagaaatgttttctcagttcgcagagtttgatgatgaactggatagcatggctccagtggggagagatgcagaaacattgcaaaagcaaaaggaaactataaaagcctttctaaagaaactagaagccctcatggcaagcaatgacaatgccaataaaacctgcaagatgatgttagccacagaagaaacctctcctgaccttgttggaatcaaaagggacttggaggccttaagcaaacaatgcaacaagttactggaccgagcccaagccagagaagagcaggttgaagggacaattaagcgccttgaagaattttacagcaaattgaaagaattttctattctgctccagaaagccgaagaacatgaagagtcacaaggtcctgttggtatggaaacggagacaattaatcagcagcttaacatgttcaaggtattccagaaagaagagattgaacccttgcaaggtaaacagcaagatgtaaactggttaggtcaaggccttattcagagtgctgccaaaagcactagcactcagggcttggagcatgacctggatgatgtcaatgcacggtggaagactctcaataagaaggtggctcagcgagcagcccagctgcaggaggccttgctgcactgtgggaggttccaggatgccctggagtccctgctcagctggatggtggacactgaggagcttgtggccaatcagaagcccccgtcggctgagttcaaagtggtaaaggcccagatacaagaacaaaagcttctccagagattgttggatgaccgaaaatctacggtggaggtaatcaaacgagaaggagaaaaaattgctacaacagcagagcccgcagataaagtgaagattttgaaacagctcagtctcttggatagcagatgggaggcattgcttaataaagctgaaacaaggaatcgtcagttggaaggtatctcggtggtagcacagcaatttcatgaaaccttagaaccactgaacgagtggcttacaaccatagaaaagaggctggtgaattgtgaacccataggaacccaagcatctaaacttgaggaacaaattgcacagcacaaagttctgcaagaggacatcttactcaggaaacaaaatgtagatcaggctttactaaatggtttagaactacttaaacaaaccacaggtgatgaagttttaataattcaagataaattggaagccattaaagcaaggtacaaagacattactaaactgagcactgatgtggccaagactctggaacaggcgctgcagcttgcaaggcggctgcactccacacacgaagagctgtgtacctggctggacaaagtggaggtggaattactttcatatgaaactcaggttctgaaaggagaagaagcaagtcaagcacaaatgagaccaaaggaactgaaaaaggaagctaagaacaacaaagccttactggactcccttaatgaagtgagcagtgctttgctggaactggtaccatggagggcaagagaaggacttgagaaaatggtagctgaggacaatgagcgctaccgattagtgagcgacaccatcactcagaaggtggaggagatcgatgcagccattctgcgatcacagcagtttgaccaagcagctgatgctgagttatcctggattactgaaacagaaaaaaaattgatgtctctgggtgacatcaggcttgagcaagaccagacttctgctcagcttcaagttcaaaagacattcaccatggagattttgagacacaaggatattattgatgaccttgttaaatctgggcataaaatcatgaccgcatgcagtgaagaggaaaagcaatcaatgaagaaaaaactggacaaggtactgaagaactatgataccatctgccagattaattcagaaaggtatctgcagctggaacgggcacagtccctggttaaccaattctgggaaacatatgaagaactttggccatggctgacagaaacacaatcaatcatctctcagcttcccgccccagcccttgaatatgaaactctaaggcagcagcaggaagaacatcggcaactgcgtgagttgatagctgaacacaagcctcatatagataagatgaacaaaactgggccacagttactggaattgagccctggggaaggcttttctatccaagagaagtatgtggcagccgacaccctttacagtcaaattaaagaagatgtcaaaaagcgtgctgtggcactggatgaagccatttctcaatcaactcagttccatgacaagatagatcagatccttgagagcctggaacgcatcgtggaacgtctgaggcagccaccctctatctctgcagaggttgagaagatcaaggaacagatcagtgaaaataagaatgtgtcagtagacatggaaaagctacagccgttgtatgaaactcttaaacagaggggagaggaaatgattgctagatctggggggactgataaagacatatctgccaaagctgttcaggataagcttgaccaaatggttttcatttgggagaacatacacacactggtggaagagagggaagccaaactactggatgtgatggagctagcagaaaagttctggtgtgatcacatgtcattgatagttaccattaaagatactcaagatttcatccgggacctggaagatcctggaattgatccttcagtagtaaaacaacagcaagaagcagcagagaccataagggaagaaatagatggactacaggaggagctggatatagttattaacctaggttctgaactcattgcggcatgtggggagcctgataaacccattgtcaagaagagtatagatgagttaaattcagcatgggattctctaaataaagcttggaaagaccggattgacaaacttgaggaggcaatgcaggctgccgttcagtaccaggatggactgcaggcggtatttgactgggtagatattgcaggtggtaaattagcttcaatgtctccaattggaacagatctcgaaactgtcaagcagcagattgaagagctaaagcaatttaagtctgaggcctatcaacagcagatagaaatggaaagactgaatcatcaagcagagcttttgctaaagaaagtaacagaagagagtgacaaacacactgttcaagacccattaatggaactgaaattgatatgggatagcctggaggagagaatcatcaacagacagcataaactggagggtgctctattagccttgggtcagttccaacatgccctggatgagctcctggcatggctgacacacaccgagggcttgctaagtgagcagaaacctgttggaggagaccctaaagccattgaaattgaacttgccaagcatcatgtgctccaaaatgatgtattagcccatcagtccacagtggaagccgttaataaagcaggaaatgatctaattgaatcaagtgcaggagaagaagcaagcaaccttcagaacaagctagaggttttaaatcaacgctggcaaaatgttttggaaaaaacagaacaaaggaagcagcagctggatggtgccttgcgccaggccaaagggttccatggcgaaattgaggatttgcagcagtggctgactgacacggagcgtcatctgttggcatctaaaccgctgggaggtttaccggaaacagccaaggagcagcttaatgtccatatggaagtctgtgctgcctttgaagctaaagaagaaacatataagagtctgatgcagaaaggccagcagatgcttgcaagatgcccaaaatctgcagagacaaatattgaccaagacataaataacttgaaagaaaaatgggaatcggtggaaaccaaactcaatgaaaggaaaactaaactggaagaggctctcaacttggcaatggagttccacaattctctccaagacttcatcaactggcttactcaggctgaacagaccctaaatgtagcttctcggccaagtctcatcttggacacagtcttatttcaaattgacgaacacaaggtttttgccaatgaagtaaattctcatcgtgagcagataatagagctggacaaaactggaacccacctaaaatattttagtcagaaacaagatgttgttctaatcaagaatctacttatcagtgtacaaagtcgatgggaaaaagtggttcaacggttggtagagagaggaagatctttggatgatgcaaggaagagagccaagcagttccatgaagcttggagtaaacttatggagtggctagaagagtcagaaaagtctttggattctgaactggaaatcgcaaatgatccagacaaaataaaaacacaacttgcacaacataaggagtttcagaaatcactcggagccaagcattctgtctacgacaccaccaacaggactggacgttctctgaaggagaaaacctccctggctgatgacaacctgaaactggatgacatgctgagtgaactcagagacaaatgggataccatatgtggaaaatctgtggaaagacaaaacaaattggaggaagccctgttattttctggacaattcacagatgccctacaggctctcattgattggttatatagagttgaaccccagctggcagaagaccagcctgttcatggagacattgatttggtgatgaatctgatcgataatcacaaggccttccaaaaagagttggggaagaggaccagcagtgtgcaggccctgaagcgctcagcccgagaactcatagaaggcagtcgggatgactcctcctgggtcaaggtccagatgcaggaattaagcacacgctgggagaccgtgtgtgcactttctatatcaaagcaaacacggttagaagcagccctgcgtcaggcagaggaattccactcggtggtacatgccctcttggagtggctggctgaggcggagcaaaccctgcgtttccatggtgtcctcccagatgatgaggatgctctccggactctcattgatcagcataaagaattcatgaagaaactggaagaaaagagagctgaactaaataaagccaccactatgggcgacaccgttttggctatctgccaccccgactccatcactaccattaagcactggataacaatcatccgggcgaggtttgaggaggtgctggcctgggcaaagcaacatcagcagagattagcaagtgctctggctgggcttattgccaaacaggaattgttggaagctttgctggcttggttgcaatgggctgaaactacacttactgataaggataaagaagtcatcccccaggagatcgaagaggtgaaagcactcattgcagaacaccagaccttcatggaggaaatgaccagaaaacagcctgatgttgataaagtaacgaagacctataagaggagagctgctgatccttcctcattacaatcccatattccagtcttggataagggacgagcaggaagaaaacgctttccagcatcaagcttgtatccctctgggtcacagacacaaattgaaaccaaaaatcctagggtaaacttactggtgagcaaatggcagcaagtctggctcctggcgttggaaagaaggaggaaactcaatgatgccttggacagactagaggagctgagggaatttgctaactttgattttgatatttggcgcaaaaaatacatgcgatggatgaatcacaagaaatctcgagtgatggacttcttcaggagaattgataaagaccaggatgggaaaataacgcggcaggaatttattgatggaattctttcctcaaagtttccaaccagtcgcttggagatgagcgcagttgcagacatctttgacagagatggcgatggatatattgactactatgaatttgtagcagcccttcacccaaataaagatgcatataaacctatcacagatgccgacaaaatcgaagatgaggtgacaaggcaggtagctaagtgtaaatgtgcaaagcgatttcaagttgagcagattggtgataataaatacaggttcttcctgggaaatcagtttggagactcccagcaactgcgactggtccggatcctgcggagtactgtgatggttcgtgttggaggtggatggatggcacttgatgagttcttagtgaaaaatgatccttgcagggccaaaggaaggacaaacatggaactgcgtgagaagttcattttagcagatggtgccagccagggtatggctgctttccgaccccgaggccgaagatcccggccatcatcacgaggcgcttcacccaacagatccacttctgtgtccagtcaggctgcgcaggcggcctccccacaggtccctgccaccaccacacccaagattctccatcctttaacacgcaattatggtaaaccatggttgacaaacagcaaaatgtcaactccttgtaaagcagcagagtgctcagactttcccgtgccatctgcagagggaacgccaatacaaggaagcaagcttcgacttccaggatatttatcagggaaaggcttccactctggggaggacagtggcttgataacaactgcagctgccagagtccgaacacagtttgctgattccaagaagactcccagccgaccaggaagtcgagctggaagcaaagctggcagcagggccagcagccgccgaggcagtgatgcatcagactttgacatttcagaaatccagtccgtgtgctcagatgtggaaactgtcccccagacacacagacctacaccccgagcaggttctcggccatccacagcgaagccttcaaaaatccccacgccccagaggaaatcacctgccagcaaattggacaagtcctcaaagagatagtgcaattggttctaccaaggcccttccttgagcatttattatttaagtttgaacgatgtaaaatatggtgtagaaattcttgtgaaatattgcaagaggcgagtttaaaattctgcagatggccttatttgtgtatttgtctttttattttatctgtataattttttttgtcagatattctggggttaaagtcacatcatatgtgaggaggaaaagtttaacatgaactaacatttctgcactgtaacgtgccgggcacacactaaactcagttactgtacctacaggtaagtctacatcctctctgacagccacagcactacatcaatccctgacgttagggatacctcatgacattttcctgtttttatggaaactctgagaagctgaatgatacatgcaggggatattttttgagatgatttaaatgtaaaccaaaagatggaagacaaaaagacaaacacacccacacgcagtctttgcagtatctgacagagaactcacaggaagttacttcaagcacttgccagtactatgatattcaagtaccttgcagcatttctctgccattgctttcaatgaggccagaggcatcctggatattagacctattatactgtaagaatataagtataaagtgcgttcatatacatgtgaggttttcttttgcttgagtggacagtagcacctgtatcattgaactcattttgtatcagagcaattttgcttgcagaaagctatgaaataaaacacgtcccttaactgc

SEQ ID NO: 4 - protein, 2835 aa, Homo sapiens
WLVEKELMVSVLGPLSIDPNMLNTQRQQVQILLQEFATRKPQYEQLTAAGQGILSRPGEDPSLRGIVKEQLAAVTQKWDSLTGQLSDRCDWIDQAIVKSTQYQSLLRSLSDKLSDLDNKLSSSLAVSTHPDAMNQQLETAQKMKQEIQQEKKQIKVAQALCEDLSALVKEEYLKAELSRQLEGILKSFKDVEQKAENHVQHLQSACASSHQFQQMSRDFQAWLDTKKEEQNKSHPISAKLDVLESLIKDHKDFSKTLTAQSHMYEKTIAEGENLLLKTQGSEKAALQLQLNTIKTNWDTFNKQVKERENKLKESLEKALKYKEQVETLWPWIDKCQNNLEEIKFCLDPAEGENSIAKLKSLQKEMDQHFGMVELLNNTANSLLSVCEIDKEVVTDENKSLIQKVDMVTEQLHSKKFCLENMTQKFKEFQEVSKESKRQLQCAKEQLDIHDSLGSQAYSNKYLTMLQTQQKSLQALKHQVDLAKRLAQDLVVEASDSKGTSDVLLQVETIAQEHSTLSQQVDEKCSFLETKLQGIGHFQNTIREMFSQFAEFDDELDSMAPVGRDAETLQKQKETIKAFLKKLEALMASNDNANKTCKMMLATEETSPDLVGIKRDLEALSKQCNKLLDRAQAREEQVEGTIKRLEEFYSKLKEFSILLQKAEEHEESQGPVGMETETINQQLNMFKVFQKEEIEPLQGKQQDVNWLGQGLIQSAAKSTSTQGLEHDLDDVNARWKTLNKKVAQRAAQLQEALLHCGRFQDALESLLSWMVDTEELVANQKPPSAEFKVVKAQIQEQKLLQRLLDDRKSTVEVIKREGEKIATTAEPADKVKILKQLSLLDSRWEALLNKAETRNRQLEGISVVAQQFHETLEPLNEWLTTIEKRLVNCEPIGTQASKLEEQIAQHKVLQEDILLRKQNVDQALLNGLELLKQTTGDEVLIIQDKLEAIKARYKDITKLSTDVAKTLEQALQLARRLHSTHEELCTWLDKVEVELLSYETQVLKGEEASQAQMRPKELKKEAKNNKALLDSLNEVSSALLELVPWRAREGLEKMVAEDNERYRLVSDTITQKVEEIDAAILRSQQFDQAADAELSWITETEKKLMSLGDIRLEQDQTSAQLQVQKTFTMEILRHKDIIDDLVKSGHKIMTACSEEEKQSMKKKLDKVLKNYDTICQINSERYLQLERAQSLVNQFWETYEELWPWLTETQSIISQLPAPALEYETLRQQQEEHRQLRELIAEHKPHIDKMNKTGPQLLELSPGEGFSIQEKYVAADTLYSQIKEDVKKRAVALDEAISQSTQFHDKIDQILESLERIVERLRQPPSISAEVEKIKEQISENKNVSVDMEKLQPLYETLKQRGEEMIARSGGTDKDISAKAVQDKLDQMVFIWENIHTLVEEREAKLLDVMELAEKFWCDHMSLIVTIKDTQDFIRDLEDPGIDPSVVKQQQEAAETIREEIDGLQEELDIVINLGSELIAACGEPDKPIVKKSIDELNSAWDSLNKAWKDRIDKLEEAMQAAVQYQDGLQAVFDWVDIAGGKLASMSPIGTDLETVKQQIEELKQFKSEAYQQQIEMERLNHQAELLLKKVTEESDKHTVQDPLMELKLIWDSLEERIINRQHKLEGALLALGQFQHALDELLAWLTHTEGLLSEQKPVGGDPKAIEIELAKHHVLQNDVLAHQSTVEAVNKAGNDLIESSAGEEASNLQNKLEVLNQRWQNVLEKTEQRKQQLDGALRQAKGFHGEIEDLQQWLTDTERHLLASKPLGGLPETAKEQLNVHMEVCAAFEAKEETYKSLMQKGQQMLARCPKSAETNIDQDINNLKEKWESVETKLNERKTKLEEALNLAMEFHNSLQDFINWLTQAEQTLNVASRPSLILDTVLFQIDEHKVFANEVNSHREQIIELDKTGTHLKYFSQKQDVVLIKNLLISVQSRWEKVVQRLVERGRSLDDARKRAKQFHEAWSKLMEWLEESEKSLDSELEIANDPDKIKTQLAQHKEFQKSLGAKHSVYDTTNRTGRSLKEKTSLADDNLKLDDMLSELRDKWDTICGKSVERQNKLEEALLFSGQFTDALQALIDWLYRVEPQLAEDQPVHGDIDLVMNLIDNHKAFQKELGKRTSSVQALKRSARELIEGSRDDSSWVKVQMQELSTRWETVCALSISKQTRLEAALRQAEEFHSVVHALLEWLAEAEQTLRFHGVLPDDEDALRTLIDQHKEFMKKLEEKRAELNKATTMGDTVLAICHPDSITTIKHWITIIRARFEEVLAWAKQHQQRLASALAGLIAKQELLEALLAWLQWAETTLTDKDKEVIPQEIEEVKALIAEHQTFMEEMTRKQPDVDKVTKTYKRRAADPSSLQSHIPVLDKGRAGRKRFPASSLYPSGSQTQIETKNPRVNLLVSKWQQVWLLALERRRKLNDALDRLEELREFANFDFDIWRKKYMRWMNHKKSRVMDFFRRIDKDQDGKITRQEFIDGILSSKFPTSRLEMSAVADIFDRDGDGYIDYYEFVAALHPNKDAYKPITDADKIEDEVTRQVAKCKCAKRFQVEQIGDNKYRFFLGNQFGDSQQLRLVRILRSTVMVRVGGGWMALDEFLVKNDPCRAKGRTNMELREKFILADGASQGMAAFRPRGRRSRPSSRGASPNRSTSVSSQAAQAASPQVPATTTPKILHPLTRNYGKPWLTNSKMSTPCKAAECSDFPVPSAEGTPIQGSKLRLPGYLSGKGFHSGEDSGLITTAAARVRTQFADSKKTPSRPGSRAGSKAGSRASSRRGSDASDFDISEIQSVCSDVETVPQTHRPTPRAGSRPSTAKPSKIPTPQRKSPASKLDKSSKR